(19) United States
(12) Patent Application Publication
(10) Pub. No.: US 2010/0209532 A1
(43) Pub. Date: Aug. 19, 2010

(54) TARGETED OXYGEN DELIVERY VIA INTRAVENOUS OR INTRA-ARTERIAL INFUSION OF OXYGENATED POLYMERIZED HEMOGLOBIN SOLUTIONS

(75) Inventors: Gregory P. Dube, Littleton, MA (US); Zafiris W. Zafirelis, Needham, MA (US); Anthony J. Laccetti, North Andover, MA (US); Javed Baqai, Lexington, MA (US)

Correspondence Address: JONES DAY, 222 EAST 41ST ST, NEW YORK, NY, 10017, US

(73) Assignee: OPK BIOTECH LLC, Cambridge, MA (US)

(21) Appl. No.: 12/451,997

(22) PCT Filed: Jun. 13, 2008
(86) PCT No.: PCT/US2008/007445

§ 371 Date: Apr. 29, 2010

Related U.S. Application Data
(60) Provisional application No. 60/934,448, filed on Jun. 13, 2007.

(51) Int. Cl. A61K 38/42; A61K 33/42; A61K 33/06; A61K 33/00; A61P 9/10

(52) U.S. Cl. 424/606; 424/682; 424/717; 514/4; 514/6

(57) ABSTRACT

A method of delivering oxygen to a tissue, a blood vessel, an organ, or a region of an organ, under an ischemic condition, or prophylactically preventing occurrence of an ischemic condition, of a subject, comprising the step of administering to the subject an oxygenated hemoglobin solution, wherein the oxygenated hemoglobin solution includes polymerized hemoglobin, and wherein about 80% by weight, or greater, of the polymerized hemoglobin is oxyhemoglobin.

### RELATED APPLICATIONS

This application claims the benefit of U.S. Provisional Application No. 60/934,448, filed on Jun. 13, 2007. The entire teachings of the above applications are incorporated herein by reference.

### INCORPORATION BY REFERENCE

The entire teachings of International Application No. PCT/US2006/012676, which designated the United States and was filed on Apr. 5, 2006, published in English, and references cited therein are incorporated herein by reference.

### BACKGROUND OF THE INVENTION

In recent years, major advances in the medical management have been made for the treatment of both acute myocardial infarction (AMI) and percutaneous coronary intervention (PCI), attributable to the introduction of effective drugs and devices, and to the significant reduction of “time to reperfusion.” Nevertheless, morbidity and mortality from AMI remain significant; over 500,000 people die each year in Europe from ischemic heart disease, including AMI and its complications, mainly as a consequence of fatal arrhythmias and congestive heart failure.

The natural progression of cardiovascular disease may ultimately interrupt blood flow to the heart (resulting in angina or acute myocardial infarction, i.e., heart attack), brain (resulting in acute brain ischemia, i.e., stroke), nerves (resulting in neuropathy) or limbs (resulting in gangrene). Most acute arterial occlusions are due to thrombotic events in arteries already compromised by some degree of fixed arterial stenosis. In addition, a variety of elective percutaneous arterial interventions require temporary interruption of blood flow to an organ or a region of an organ, including elective or prophylactic percutaneous arterial interventions (angioplasty, stent deployment, atherectomy, angiography, angioscopy and optical coherence tomography imaging [OCT]). Numerous surgical interventions also require interruption of arterial blood flow including coronary artery bypass surgery, peripheral artery bypass grafting, aneurysm repair, carotid endarterectomy, aortic surgery (particularly that which interrupts flow to the kidneys) and revascularization of the mesenteric blood supply (superior mesenteric artery syndrome with intestinal angina). Trauma surgery and organ transplant are also associated with arterial cross-clamping that subjects organs to ischemia. Approximately 1,000,000 percutaneous coronary interventions are performed annually in both the United States and Europe.

During these interruptions, downstream tissues often become transiently ischemic and, in some cases, may sustain irreversible damage. Following coronary occlusion and oxygen deprivation, myocardial necrosis begins within 15 minutes and, without any intervention, results in irreversible damage over the next 30 to 90 minutes. Even if not permanently compromised, the downstream ischemic region may require considerable time to fully recover, during which the patient has compromised organ function. Previous studies have shown that in acute myocardial infarction, the risk of both myocardial transmural necrosis and severe microvascular obstruction increases with the duration of ischemia (see Coccolini S, et al., Ital Heart J 2003; 4(2 Suppl):102-11; and Andersen et al., N Engl J Med 2003; 349(8):733-42, the entire teachings of all of which are incorporated herein by reference).

Facilitation of coronary intervention with “upstream” cardio-protective interventions and drugs able to enhance the viability of cardiac myocytes and reduce the amount of necrosis may result in better clinical outcomes in the setting of AMI or acute coronary syndromes. Successful myocardial revascularization performed early in the period of acute regional ischemia may avoid extensive myocardial necrosis and, therefore, preserve left ventricular function. Early intervention that may include embolectomy, thrombolytic therapy or percutaneous arterial interventions that revascularizes the affected organ results in salvage of ischemic tissue at risk of becoming irreversibly damaged, reducing the mass of tissue that would otherwise become permanently dysfunctional. Currently, percutaneous revascularization interventions are generally performed in the absence of intra-arterial infusions and, consequently, are often associated with transient ischemia of downstream tissue. For example, OCT imaging requires that RBCs be displaced from the field of view to allow penetration of the near-infrared light. This is accomplished by intracoronary infusion of normal saline a medium that not only carries little oxygen, but which also perturbs local trans-sarcolemmal ion gradients (see de Smet, BJGL and Zijlstra F. A look at drug eluting stents with optical coherence tomography. Eur Heart J. 28: 918-919, 2007, the entire teachings of which are incorporated herein by reference). In the heart, this significantly increases the risk of angina and life-threatening cardiac arrhythmias.

Revascularization that includes perfusion of the ischemic organ with a plegic solution may be more efficacious in salvaging ischemic tissue at risk than restoration of blood flow alone. Vital organs particularly impacted by occlusive vascular disease, and potentially protected by plegic solutions, include, but are not limited to the heart, brain, kidneys and liver (see Kumbhani D J, Sharma G V, Khuri S F, Kirdar J A. Fascicular conduction disturbances after coronary artery bypass surgery: a review with a meta-analysis of their long-term significance. J Card Surg. 21:428-234, 2006; Harrington D K, Fragomeni F, Bonser R S. Cerebral perfusion. Ann Thorac Surg. 83:S799-804, 2007; discussion S824-31; and Feng X N, Xu X, Zheng S S. Current status and perspective of liver preservation solutions. Hepatobiliary Pancreat Dis Int. 5:490-494, 2006, the entire teachings of all of which are incorporated herein by reference).

Various cardioplegic infusion solutions have been developed to improve tissue preservation during the induced cardiac arrest and no-flow period required for cardiac surgery (e.g., coronary artery bypass grafting (CABG) and valve replacement) (See Donnelly A J, Djuric M. Cardioplegia solutions. Am J Hosp Pharm, 48:2444-2460, 1991; Feindel C M, Tait G A, Wilson G J, Klement P, MacGregor D C. Multidose blood versus crystalloid cardioplegia. Comparison by quantitative assessment of irreversible myocardial injury. J Thorac Cardiovasc Surg. 87:585-95, 1984; Stocker C F, Shekerdemian L S. Recent developments in the perioperative management of the pediatric cardiac patient. Curr Opin Anaesthesiol. 19:375-381, 2006; and Daggett W M Jr, Randolph J D, Jacobs M, O'Keefe D D, Geffin G A, Swinski L A, Boggs B R, Austen W G. The superiority of cold oxygenated dilute blood cardioplegia. Ann Thorac Surg. 43:397-402, 1987, the entire teachings of all of which are incorporated herein by reference). Putatively optimized crystalloid cardioplegic solutions, containing physiologic ions and a variety of other agents that depend on the particular institution and surgical team, but commonly include insulin, glucose and elevated potassium have been the standard-of-care for decades.

Despite a long history of preclinical and clinical research to improve cardioplegic solutions, suboptimal post-surgical cardiac recovery and associated mortality remain some of the most challenging issues in cardiac surgery. Infusing cardioplegic solutions into the ischemic zone via the infarct-related artery have also been studied preclinically as adjunct therapies to minimize infarct size to treat acute myocardial ischemia associated with unstable angina, but are not in clinical use for this indication. In unstable angina patients, cardioplegic access to remote areas of the infarct-related coronary bed may be difficult due to upstream coronary constriction and the well-known microvascular-related no-reflow phenomenon upon restoration of conduit artery patency (see Ito H. No-reflow phenomenon and prognosis in patients with acute myocardial infarction. Nat Clin Pract Cardiovasc Med 3:499-506, 2006; and Bolognese L, Falsini G, Liistro F, Angioli P, Ducci K. Epicardial and microvascular reperfusion with primary percutaneous coronary intervention. Ital Heart J. 6:447-452, 2005, the entire teachings of all of which are incorporated herein by reference).

Therefore, there is a need for a product and method for protecting cardiac function in the absence of coronary blood flow, which can reduce or eliminate one or more of the above-mentioned problems.

### SUMMARY OF THE INVENTION

Generally, the invention is directed to delivery of an oxygenated hemoglobin solution to an organ or organism under an ischemic condition.

In one embodiment, the invention includes a method of delivering oxygen to a tissue, a blood vessel, an organ, a region of an organ or an organism under an ischemic condition of a subject, by administering to the subject an oxygenated hemoglobin solution, wherein the oxygenated hemoglobin solution includes polymerized hemoglobin, and wherein about 80% by weight, or greater, of the polymerized hemoglobin is oxyhemoglobin.

In another embodiment, the invention includes a method of treating a patient having ischemia or angina, by administering to the subject an oxygenated hemoglobin solution, wherein the oxygenated hemoglobin solution includes polymerized hemoglobin, and wherein about 80% by weight, or greater, of the polymerized hemoglobin is oxyhemoglobin.

In still another embodiment, the invention is a plegic solution, appropriate for perfusing one or more different organ types, including a physiological buffer having a pH between about 7.6 and about 7.9; glucose; and polymerized hemoglobin in an amount of between about 10 grams per liter of solution and about 250 grams per liter of solution. The polymerized hemoglobin is about 80% by weight, or greater, oxyhemoglobin, about 18% by weight, or less, has a molecular weight of over 500,000 Daltons, about 5% by weight, or less, has a molecular weight equal to or less than 65,000 Daltons, and a P₅₀ is in a range of between about 34 and about 46 mm Hg. The endotoxin content of the plegic solution is less than about 0.05 endotoxin units per milliliter.

In another embodiment, the invention is an oxygenated hemoglobin solution, as described herein, for use in treating a patient having ischemia or angina, wherein the oxygenated hemoglobin solution includes polymerized hemoglobin, and wherein about 80% by weight, or greater, of the polymerized hemoglobin is oxyhemoglobin.

In a further embodiment, the invention is an oxygenated hemoglobin solution, as described herein, packaged and presented for use in treating a patient having ischemia or angina, wherein the oxygenated hemoglobin solution includes polymerized hemoglobin, and wherein about 80% by weight, or greater, of the polymerized hemoglobin is oxyhemoglobin.

In a skill further embodiment, the invention is the use of an oxygenated hemoglobin solution, as described herein, for the manufacture of a medicament for treating a patient having ischemia or angina, wherein the oxygenated hemoglobin solution includes polymerized hemoglobin, and wherein about 80% by weight, or greater, of the polymerized hemoglobin is oxyhemoglobin.

The method of the invention can restore oxygenation to ischemic tissue. The physiochemical and biochemical properties of oxygenated polymerized hemoglobin solution, such as HEMOPURE® (also known as HBOC-201) hemoglobin glutamer-250 (bovine), hemoglobin-based oxygen carrier (HBOC) (low viscosity, molecular size, P₅₀), restores tissue oxygenation and protects histological and functional integrity of the target tissue(s), when infused into the arterial circulation of an ischemic organ or via retrograde infusion into the venous circulation of an ischemic organ or into the central venous circulation of an organism. Hemoglobin solution suitable for use in the method of the invention can be stored at room temperature for up to 3 years, is free of blood-born infectious agents and does not need to be cross-matched. Although not to be held to any particular theory, it is believed that treatment of ischemic tissue by the method of the invention improves tissue oxygenation by delivering oxygen to post-stenotic areas that free plasma, but not red blood cells, are capable of reaching.

### BRIEF DESCRIPTION OF THE DRAWINGS

FIGS. 1A and 1B are schematic diagrams showing one embodiment of an oxygenation system for preparing an oxygenated hemoglobin solution of the invention.

FIG. 2 is a graph of the measured spectralon reflectance versus depth for saline and HEMOPURE® HBOC. Attenuation includes focal losses. To obtain net attenuation coefficients, subtract D₂O losses: for saline μₜ=1.09−0.86 mm⁻¹=0.023 mm⁻¹ and for Hemopure HBOC μₜ=0.95−0.86 mm⁻¹=0.009.

FIG. 3 is a schematic drawing showing study evaluation time points for tests to evaluate hemoglobin based oxygen therapeutics in elective percutaneous coronary revascularization in man.

FIG. 4 is a schematic drawing showing a short over-the-wire (OTW) balloon positioned inside a stented segment to perform a selective intracoronary infusion of oxygenated HEMOPURE® HBOC to the region of interest.

FIGS. 5A and 5B are graphs showing pressure-volume loop of a representative subject (5A) during control occlusion (no intracoronary infusion) and (5B) during test occlusion with simultaneous intracoronary infusion of oxygenated HEMOPURE® HBOC.

FIGS. 6A and 6B are graphs showing (6A) left ventricular end-diastolic pressure and (6B) cardiac output of a representative subject during control occlusion (no intracoronary infusion) and during test occlusion with simultaneous intracoronary infusion of oxygenated HEMOPURE® HBOC. *Occlusion stopped prematurely during the control.

### DETAILED DESCRIPTION OF THE INVENTION

The foregoing will be apparent from the following more particular description of example embodiments of the invention, as illustrated in the accompanying drawings. The drawings are not necessarily to scale, emphasis instead being placed upon illustrating embodiments of the present invention. All references, literature, patents or other references are incorporated herein in their entirety.

The invention generally is directed to a method of delivering oxygen to a tissue, a blood vessel, an organ, a region of an organ or an organism under an ischemic condition or to a patient having angina. In one embodiment, the method includes administering to the subject an oxygenated hemoglobin solution, wherein the oxygenated hemoglobin solution includes polymerized hemoglobin, and where about 80% by weight, or greater, of the polymerized hemoglobin is oxyhemoglobin.

In the invention, the hemoglobin solution employed is typically derived from a deoxygenated hemoglobin solution, which is oxygenated prior to administration to an organism, tissue, blood vessel, organ, or region of an organ under an ischemic condition or to a patient having angina according to the method of the invention. As used herein, the term “deoxygenated hemoglobin solution” means that in the solution, the content of oxyhemoglobin is less than about 10% by weight based on the total hemoglobin. Preferably, the deoxygenated hemoglobin solutions that include polymerized hemoglobin are oxygenated by the oxygenation methods of the invention to have at least about 80% oxyhemoglobin by weight based on the total hemoglobin, more preferably at least about 90% oxyhemoglobin by weight based on the total hemoglobin. In a specifically preferred embodiment, the deoxygenated hemoglobin solutions that include polymerized hemoglobin are oxygenated at the aforementioned hemoglobin-solution and oxygen-gas flow rates through a hydrophobic hollow fiber cartridge having the aforementioned surface areas, to have at least about 80% oxyhemoglobin by weight based on the total hemoglobin, more preferably at least about 90% oxyhemoglobin by weight based on the total hemoglobin.

Polymerized hemoglobin that can be used in preparing the oxyhemoglobin solutions employed by the method of the invention can be prepared by procedures known in the art, including red blood cell (RBC) collection, purification of the RBC, hemoglobin polymerization and purification of the polymerized hemoglobin. Typically, during the procedures, the blood solution, RBCs and hemoglobin are maintained under conditions sufficient to minimize microbial growth, or bioburden, such as maintaining temperature at less than about 20° C. and above 0° C. Detailed descriptions about the preparation and purification of polymerized hemoglobin (Hb) solutions suitable for the invention can be found in U.S. Pat. Nos. 5,084,558; 5,955,581; 5,753,616; 5,854,209; 5,691,453; 5,691,452; 5,808,011; 5,952,470; 5,895,810; and 5,840,852, the entire teachings of which are incorporated herein by reference.

Suitable RBC sources include human blood, bovine blood, ovine blood, porcine blood, blood from other vertebrates and transgenically-produced hemoglobin, such as the transgenic Hb described in Biotechnology, 12: 55-59 (1994), the teachings of which are incorporated herein by reference. Preferably, the RBC source is bovine.

The blood can be collected from live or freshly slaughtered non-human donors. One method for collecting bovine whole blood is described in U.S. Pat. Nos. 5,084,558 and 5,296,465, the entire teachings of which are incorporated herein by reference.

In one example, at or soon after collection, the blood is mixed with at least one anticoagulant to prevent significant clotting of the blood. Suitable anticoagulants for blood are as classically known in the art and include, for example, sodium citrate, ethylenediaminetetraacetic acid and heparin. When mixed with blood, the anticoagulant may be in a solid form, such as a powder, or in an aqueous solution.

The blood solution source can be from a freshly collected sample or from an old sample, such as expired human blood from a blood bank. Further, the blood solution could previously have been maintained in frozen and/or liquid state.

Optionally, prior to introducing the blood solution to anticoagulants, antibiotic levels in the blood solution, such as penicillin, are assayed. Antibiotic levels are determined to provide a degree of assurance that the blood sample is not burdened with an infecting organism by verifying that the donor of the blood sample was not being treated with an antibiotic. Examples of suitable assays for antibiotics include a penicillin assay kit (Difco, Detroit, Mich.) employing a method entitled “Rapid Detection of Penicillin in Milk”. It is preferred that blood solutions contain a penicillin level of less than or equal to about 0.008 units/ml. Alternatively, a herd management program to monitor the lack of disease in or antibiotic treatment of the cattle may be used.

Preferably, the blood solution is strained prior to or during the anticoagulation step, for example by straining, to remove large aggregates and particles. A 600 mesh screen is an example of a suitable strainer.

The RBCs in the blood solution are then washed by suitable means, such as by diafiltration or by a combination of discrete dilution and concentration steps with at least one solution, such as an isotonic solution, to separate RBCs from extracellular plasma proteins, such as serum albumins or antibodies (e.g., immunoglobulins (IgG)). It is understood that the RBCs can be washed in a batch or continuous feed mode.

Acceptable isotonic solutions are as known in the art and include solutions, such as a citrate/saline solution, having a pH and osmolality which does not rupture the cell membranes of RBCs and which displaces the plasma portion of the whole blood. A preferred isotonic solution has a neutral pH and an osmolality between about 285-315 mOsm. A preferred isotonic solution is composed of an aqueous solution of sodium citrate dihydrate (6.0 g/l) and of sodium chloride (8.0 g/L).

Water which can be used in the method of invention includes distilled water, deionized water, water-for-injection (WFI) and/or low pyrogen water (LPW). WFI, which is preferred, is deionized, distilled water that meets U.S. Pharmacological Specifications for water-for-injection. WFI is further described in Pharmaceutical Engineering, 11, 15-23 (1991). LPW, which is preferred, is deionized water containing less than 0.002 EU/ml.

The isotonic solution can be filtered prior to being added to the blood solution. Examples of suitable filters include a Millipore 10,000 Dalton ultrafiltration membrane, such as a Millipore Cat #CDUF 050 G1 filter or A/G Technology hollow fiber, 10,000 Dalton (Cat # UFP-1O-C-85).

RBCs in the blood solution can be washed by diafiltration. Suitable diafilters include microporous membranes with pore sizes which will separate RBCs from substantially smaller blood solution components, such as a 0.1 μm to 0.5 μm filter (e.g., a 0.2 μm hollow fiber filter, Microgon Krosflo II microfiltration cartridge). Concurrently, a filtered isotonic solution is added continuously (or in batches) as makeup at a rate equal to the rate (or volume) of filtrate lost across the diafilter. During RBC washing, components of the blood solution which are significantly smaller in diameter than RBCs, or are fluids such as plasma, pass through the walls of the diafilter in the filtrate. RBCs, platelets and larger bodies of the diluted blood solution, such as white blood cells, are retained and mixed with isotonic solution, which is added continuously or batchwise to form a dialyzed blood solution.

Alternatively, the RBCs can be washed through a series of sequential (or reverse sequential) dilution and concentration steps, wherein the blood solution is diluted by adding at least one isotonic solution, and is concentrated by flowing across a filter, thereby forming a dialyzed blood solution.

RBC washing is complete when the level of plasma proteins contaminating the RBCs has been substantially reduced (typically at least about 90%). Typically, RBC washing is complete when the volume of filtrate drained from diafilter 34 equals about 300%, or more, of the volume of blood solution contained in the diafiltration tank prior to diluting the blood solution with filtered isotonic solution. Additional RBC washing may further separate extracellular plasma proteins from the RBCs. For instance, diafiltration with 6 volumes of isotonic solution may remove at least about 99% of IgG from the blood solution.

The dialyzed blood solution is then exposed to means for separating the RBCs in the dialyzed blood solution from the white blood cells and platelets, such as by centrifugation.

It is understood that other methods generally known in the art for separating RBCs from other blood components can also be employed. For example, sedimentation, wherein the separation method does not rupture the cell membranes of a significant amount of the RBCs, such as less than about 30% of the RBCs, prior to RBC separation from the other blood components.

Following separation of the RBCs, the RBCs are lysed by a means for lysing RBCs to release hemoglobin from the RBCs to form a hemoglobin-containing solution. Lysis means can use various lysis methods, such as mechanical lysis, chemical lysis, hypotonic lysing or other known lysing methods which release hemoglobin without significantly damaging the ability of the Hb to transport and release oxygen.

When recombinantly produced hemoglobin is used, the bacteria cells containing the hemoglobin are washed and separated from contaminants as described above. These bacteria cells are then mechanically ruptured by means known in the art, such as a ball mill, to release hemoglobin from the cells and to form a lysed cell phase. This lysed cell phase is then processed as is the lysed RBC phase.

The oxygenated hemoglobin solutions of the invention preferably have levels of endotoxins, phospholipids, foreign proteins and other contaminants which will not result in a significant immune system response and which are non-toxic to the recipient. Preferably, the oxygenated hemoglobin solutions of the invention are ultrapure. Ultrapure as defined herein, means containing less than 0.05 EU/ml of endotoxin, less than 3.3 nmoles/ml phospholipids and little to no detectable levels of non-hemoglobin proteins, such as serum albumin or antibodies.

As used herein, the term “endotoxin(s)” means the generally cell-bound lipopolysaccharides produced as a part of the outer layer of gram-negative bacterial cell walls, which under many conditions are toxic. When administered to animals, endotoxins can cause fever, diarrhea, hemorrhagic shock and other tissue damages. By the term “endotoxin unit” (EU) is intended that meaning given by the United States Pharmacopeial Convention of 1983, Page 3014, which defined EU as the activity contained in 0.2 nanograms of the U.S. reference standard lot EC-2. One vial of EC-2 contains 5,000 EU. Preferably, an endotoxin content of the oxygenated hemoglobin solutions of the invention is less than about 0.5 endotoxin units per milliliter, such as less than about 0.25 endotoxin units per milliliter, less than about 0.05 endotoxin units per milliliter, or less than about 0.02 endotoxin units per milliliter. The endotoxin contents can be measured, for example, by the Limulus Amebocytic Lysate (LAL) assay known in the art.

Following lysis, the lysed RBC phase is then ultrafiltered to remove larger cell debris, such as proteins with a molecular weight above about 100,000 Daltons. Generally, cell debris include all whole and fragmented cellular components with the exception of Hb, smaller cell proteins, electrolytes, coenzymes and organic metabolic intermediates. Acceptable ultrafilters include, for example, 100,000 Dalton filters made by Millipore (Cat #CDUF 050H 1) and made by AJQ Technology (Needham, Mass.; Model No. UFP100E55).

The concentrated Hb solution can then be directed into one or more parallel chromatographic columns to further separate the hemoglobin by high performance liquid chromatography from other contaminants such as antibodies, endotoxins, phospholipids and enzymes and viruses. Examples of suitable media include anion exchange media, cation exchange media, hydrophobic interaction media and affinity media. Specific examples of the suitable media include an anion exchange medium suitable to separate Hb from non-hemoglobin proteins. Suitable anion exchange mediums include, for example, silica, alumina, titania gel, cross-linked dextran, agarose or a derivatized moiety, such as a polyacrylamide, a polyhydroxyethyl-methacrylate or a styrene divinylbenzene, that has been derivatized with a cationic chemical functionality, such as a diethylaminoethyl or quaternary aminoethyl group. A suitable anion exchange medium and corresponding eluants for the selective absorption and desorption of Hb as compared to other proteins and contaminants, which are likely to be in a lysed RBC phase, are readily determinable by one of reasonable skill in the art.

Optionally, a method can be used to form an anion exchange media from silica gel, which is hydrothermally treated to increase the pore size, exposed to 7-glycidoxy propylsilane to form active epoxide groups and then exposed to 03H₇(CHs)NCl to form a quaternary ammonium anion exchange medium. This method is described in the Journal of Chromatography, 120:321-333 (1976), which is incorporated herein by reference in its entirety.

In one specific example, chromatographic columns are first pre-treated by flushing with a first eluant which facilitates Hb binding. Concentrated Hb solution is then injected onto the medium in the columns. After injecting the concentrated Hb solution, the chromatographic columns are then successively washed with different eluants to produce a separate, purified Hb eluate.

Generally, a pH gradient is used in chromatographic columns to separate protein contaminants, such as the enzyme-carbonic anhydrase, phospholipids, antibodies and endotoxins from the Hb. Each of a series of buffers having different pH values, are sequentially directed to create a pH gradient within the medium in the chromatographic column. The use of pH gradients to separate Hb form non-hemoglobin contaminants is further described in U.S. Pat. No. 5,691,452, filed Jun. 7, 1995, which are incorporated herein by reference.

An example of the first buffer is a tris-hydroxymethyl aminomethane (Tris) solution (concentration about 20 mM; pH about 8.4 to about 9.4). An example of the second buffer is a mixture of the first buffer and a third buffer, with the second buffer having a pH of about 8.2 to about 8.6. An example of the third buffer is a Tris solution (concentration about 50 mM; pH about 6.5 to about 7.5). An example of the fourth buffer is a NaCl/Tris solution (concentrations about 1.0 M NaCl and about 20 mM Tri; pH about 8.4 to about 9.4, preferably about 8.9-9.1).

Typically, the buffers used are at a temperature between about 0° C. and about 50° C. Preferably, buffer temperature is about 12.4±1.0° C. during use. In addition, the buffers are typically stored at a temperature of about 9° C. to about 11° C. The Hb eluate is then preferably deoxygenated prior to polymerization to form a deoxygenated Hb solution by means that substantially deoxygenate the Hb without significantly reducing the ability of the Hb in the Hb eluate to transport and release oxygen, such as would occur from denaturation of formation of oxidized hemoglobin (metHb).

The deoxygenated-Hb is then preferably equilibrated with a low oxygen content storage buffer, containing a sulfhydryl compound, to form an oxidation-stabilized deoxygenated Hb. Suitable sulfhydryl compounds include non-toxic reducing agents, such as N-acetyl-L-cysteine (NAC) D,L-cysteine, γ-glutamyl-cysteine, glutathione, 2,3-dimercapto-1-propanol, 1,4-butanedithiol, thioglycolate, and other biologically compatible sulfhydryl compounds. The oxygen content of a low oxygen content storage buffer must be low enough not to significantly reduce the concentration of sulfhydryl compound in the buffer and to limit oxyhemoglobin content in oxidation stabilized deoxygenated Hb to about 20% or less, preferably less than about 10%. Typically, the storage buffer has a pO₂ of less than about 50 torr.

The amount of a sulfhydryl compound mixed with the deoxygenated Hb is an amount high enough to increase intramolecular cross-linking of Hb during polymerization and low enough not to significantly decrease intermolecular cross-linking of Hb molecules, due to a high ionic strength. Typically, about one mole of sulfhydryl functional groups (—SH) are needed to oxidation stabilize between about 0.25 moles to about 5 moles of deoxygenated Hb.

Optionally, prior to transferring the oxidation-stabilized deoxygenated Hb to a polymerization reactor, an appropriate amount of water is added to the polymerization reactor.

The pO₂ of the water in the polymerization step is generally reduced to a level sufficient to limit HbO₂ content to about 20%, typically less than about 50 torr. And then the polymerization reactor is blanketed with an inert gas, such as nitrogen. The oxidation-stabilized deoxygenated Hb is then transferred into the polymerization reactor, which is concurrently blanketed with an appropriate flow of an inert gas.

The temperature of the oxidation-stabilized deoxygenated Hb solution in polymerization reactor is raised to a temperature to optimize polymerization of the oxidation-stabilized deoxygenated Hb when contacted with a cross-linking agent. Typically, the temperature of the oxidation-stabilized deoxygenated Hb is about 25° C. to about 45° C., and preferably about 41° C. to about 43° C. throughout polymerization.

The oxidation-stabilized deoxygenated Hb is then exposed to a suitable cross-linking agent at a temperature sufficient to polymerize the oxidation-stabilized deoxygenated Hb to form a solution of polymerized hemoglobin (poly(Hb)) over a period of about 2 hours to about 6 hours.

The term “polymerized,” as used herein, encompasses both inter-molecular and intramolecular polyhemoglobin, with at least 50%, preferably greater than about 95%, of the polymerized hemoglobin of greater than tetrameric form. The polymerized hemoglobin that can be employed for the invention can be prepared by polymerizing or cross-linking with a multifunctional cross-linking agent. Preferably, the polymerized hemoglobin is substantially soluble in aqueous fluids having a pH of 6 to 9 and in physiological fluids.

Suitable examples of cross-linking agents are disclosed in U.S. Pat. No. 4,001,200, the entire teachings of which are incorporated herein by reference. Suitable specific examples of the cross-linking agents include compounds having an aldehyde or dialdehyde functionality, such as formaldehyde, paraformaldehyde, formaldehyde activated ureas such as 1,3-bis(hydroxymethyl)urea, N,N′-di(hydroxymethyl) imidazolidinone prepared from formaldehyde condensation with a urea; compounds bearing a functional isocyanate or isothiocyanate group, such as diphenyl-4,4′-diisothiocyanate-2,2′-disulfonic acid, toluene diisocyanate, toluene-2-isocyanate-4-isothiocyanate, 3-methoxydiphenylmethane-4,4′-diisocyanate, propylene diisocyanate, butylene diisocyanate, and hexamethylene diisocyanate; esters and thioesters activated by strained thiolactones; hydroxysuccinimide esters; halogenated carboxylic acid esters; and imidates. Other examples of the cross-linking agents include derivatives of carboxylic acids and carboxylic acid residues of hemoglobin activated in situ to give a reactive derivative of hemoglobin that will cross-link with the amines of another hemoglobin. Examples of the carboxylic acids include citric, malonic, adipic and succinic acids. Carboxylic acid activators include thionyl chloride, carbodiimides, N-ethyl-5-phenyl-isoxazolium-3′-sulphonate (Woodward's reagent K), N,N′-carbonyldiimidazole, N-t-butyl-5-methylisoxazolium perchlorate (Woodward's reagent L), 1-ethyl-3-dimethyl aminopropylcarbodiimde, and 1-cyclohexyl-3-(2-morpholinoethyl) carbodiimide metho-p-toluene sulfonate. The cross-linking reagent can be a dialdehyde precursor that readily forms a bifunctional dialdehyde in the reaction medium. Suitable dialdehyde precursors include acrolein dimer or 3,4-dihydro-1,2-pyran-2-carboxaldehyde which undergoes ring cleavage in an aqueous environment to give alpha-hydroxy-adipaldehyde. Other precursors, which on hydrolysis yield a cross-linking reagent, include 2-ethoxy-3,4-dihydro-1,2-pyran which gives glutaraldehyde, 2-ethoxy-4-methyl-3,4-dihydro-1,2-pyran which yields 3-methyl glutaraldehyde, 2,5-diethoxy tetrahydrofuran which yields succinic dialdehyde and 1,1,3,3-tetraethoxypropane which yields malonic dialdehyde and formaldehyde from trioxane. Exemplary commercially-available cross-linking reagents include divinyl sulfone, epichlorohydrin, butadiene diepoxide, ethylene glycol diglycidyl ether, glycerol diglycidyl ether, dimethyl suberimidate dihydrochloride, dimethyl malonimidate dihydrochloride, and dimethyl adipimidate dihydrochloride.

Preferred specific examples of the cross-linking agents include glutaraldehyde, succindialdehyde, activated forms of polyoxyethylene and dextran, α-hydroxy aldehydes, such as glycolaldehyde, N-maleimido-6-aminocaproyl-(2′-nitro, 4′-sulfonic acid)-phenyl ester, m-maleimidobenzoic acid-N-hydroxysuccinimide ester, succinimidyl 4-(N-maleimidomethyl)cyclohexane-1-carboxylate, sulfosuccinimidyl 4-(N-maleimidomethyl)cyclohexane-1-carboxylate, m-maleimidobenzoyl-N-hydroxysuccinimide ester, m-maleimidobenzoyl-N-hydroxysulfosuccinimide ester, N-succinimidyl(4-iodoacetyl)aminobenzoate, sulfosuccinimidyl(4-iodoacetyl)aminobenzoate, succinimidyl 4-(p-maleimidophenyl)butyrate, sulfosuccinimidyl 4-(p-maleimidophenyl)butyrate, 1-ethyl-3-(3-dimethylaminopropyl)carbodiimide hydrochloride, N,N′-phenylene dimaleimide, and compounds belonging to the bis-imidate class, the acyl diazide class or the aryl dihalide class.

Preferred polymerized hemoglobin that can be employed in the invention includes hemoglobin polymerized by a dialdehyde. As used herein, the “hemoglobin polymerized by a dialdehyde” includes both hemoglobin polymerized by a dialdehyde and hemoglobin polymerized by a dialdehyde precursor that readily forms a bifunctional dialdehyde in the reaction medium. Suitable dialdehyde and dialdehyde precursors are as described above. More preferred polymerized hemoglobin that can be employed in the invention includes hemoglobin polymerized by glutaraldehyde. In some embodiments, the oxygenated hemoglobin solutions of the invention include hemoglobin polymerized by glutaraldehyde, but not pyridoxylated by a pyridoxylating agent, such as pyridoxal 5′ phosphate.

In a specific example, glutaraldehyde is used as the cross-linking agent. Typically, about 10 to about 70 grams of glutaraldehyde are used per kilogram of oxidation-stabilized deoxygenated Hb. In a more specific example, glutaraldehyde is added over a period of five hours until approximately 29-31 grams of glutaraldehyde are added for each kilogram of oxidation-stabilized deoxygenated Hb.

A suitable amount of a cross-linking agent is that amount which will permit intramolecular cross-linking to stabilize the Hb and also intermolecular cross-linking to form polymers of Hb, to thereby increase intravascular retention. Typically, a suitable amount of a cross-linking agent is that amount wherein the molar ratio of cross-linking agent to Hb is in excess of about 2:1. Preferably, the molar ratio of cross-linking agent to Hb is between about 20:1 to 40:1.

In a specific example, the polymerization is performed in a buffer with a pH between about 7.6 to about 7.9, having a chloride concentration less than or equal to about 35 mmolar.

Poly(Hb) generally has significant intramolecular cross-linking if a substantial portion (e.g., at least about 50%) of the Hb molecules are chemically bound in the poly(Hb), and only a small amount, such as less than about 10% are contained within high molecular weight polymerized hemoglobin chains. High molecular weight poly(Hb) molecules are molecules, for example, with a molecular weight above about 500,000 Daltons.

After polymerization, the temperature of the poly(Hb) solution in the polymerization reactor is typically reduced to about 15° C. to about 25° C.

Wherein the cross-linking agent used is not an aldehyde, the poly(Hb) formed is generally a stable poly(Hb). Wherein the cross-linking agent used is an aldehyde, the poly(Hb) formed is generally not stable until mixed with a suitable reducing agent to reduce less stable bonds in the poly(Hb) to form more stable bonds. Examples of suitable reducing agents include sodium borohydride, sodium cyanoborohydride, sodium dithionite, trimethylamine, t-butylamine, mopholine borane and pyridine borane. Prior to adding the reducing agent, the poly(Hb) solution is optionally concentrated by ultrafiltration until the concentration of the poly(Hb) solution is increased to between about 75 and about 85 g/L. Suitable ultrafilters are of cartridge construction designed for multiple reuse, rated at 30,000 kilodalton (kD) and contain regenerated cellulose supported membrane (e.g., Millipore Helicon, Cat #CDUF050LT and Amicon, Cat #540430).

The pH of the poly(Hb) solution is then adjusted to the alkaline pH range to preserve the reducing agent and to prevent hydrogen gas formation, which can denature Hb during the subsequent reduction. In one embodiment, the pH is adjusted to greater than 10. The pH can be adjusted by adding a buffer solution to the poly(Hb) solution during or after polymerization. The poly(Hb) is typically purified to remove non-polymerized (i.e. low molecular weight hemoglobin having less than about 65 kD) hemoglobin from higher molecular weight polymerized hemoglobin. This fractionation can be accomplished by diafiltration or hydroxyapatite chromatography (see, e.g., U.S. Pat. No. 5,691,453, which is incorporated herein by reference). Examples of commercially available 100 kD ultrafiltration membranes suitable for performing polymerized hemoglobin fractionation include Pall's 100 kD Omega polyethersulfone Amersham's polyethersulfone Kvick Flow Process Scale and Millipore's PLCHK composite regenerated cellulose.

Following the pH adjustment, at least one reducing agent, preferably a sodium borohydride solution, is added to the poly(Hb) solution. Typically, about 5 to about 18 moles of reducing agent are added per mole of Hb tetramer (per 64,000 Daltons of Hb) within the poly(Hb).

The pH and electrolytes of the stable poly(Hb) can then be restored to physiologic levels to form a stable polymerized hemoglobin solution, by diafiltering the stable poly(Hb) with a diafiltration solution having a suitable pH and physiologic electrolyte levels.

Wherein the poly(Hb) was reduced by a reducing agent, the diafiltration solution has an acidic pH, preferably between about 4 to about 6.

A non-toxic sulfhydryl compound can also be added to the stable poly(Hb) solution as an oxygen scavenger to enhance the stability of the final polymerized hemoglobin blood-substitute. The sulfhydryl compound can be added as part of the diafiltration solution and/or can be added separately. An amount of sulfhydryl compound is added to establish a sulfhydryl concentration which will scavenge oxygen to maintain methemoglobin content less than about 15% over the storage period. Preferably, the sulfhydryl compound is NAC. Typically, the amount of sulfhydryl compound added is an amount sufficient to establish a sulfhydryl concentration between about 0.05% and about 0.2% by weight.

Preferably, the polymerized hemoglobin solutions that can be used in the invention include stable polymerized hemoglobin. As used herein, the “stable polymerized hemoglobin” is a hemoglobin-based oxygen carrying composition which does not substantially increase or decrease in molecular weight distribution and/or in methemoglobin content during storage periods at suitable storage temperatures for periods of two years or more, and preferably for periods of two years or more, when stored in a low oxygen environment. Suitable storage temperatures for storage of one year or more are between about 2° C. and about 40° C.

The polymerized Hb solutions are generally packaged under aseptic handling conditions while maintaining pressure with an inert, substantially oxygen-free atmosphere, in the polymerization reactor and remaining transport apparatus. Such polymerized Hb solutions can then be used for preparing oxygenated Hb solutions of the invention by the methods described above, for example, by the use of oxygenation system 10.

Any suitable methods known in the art can be used for oxygenating in vitro the hemoglobin solutions described above. In a preferred embodiment, the hemoglobin solutions are oxygenated in vitro with the use of a filter in a single flow-through, whereby an oxygen gas makes contact with the hemoglobin solution within the hydrophobic pores of the filter, diffuses into the hemoglobin solution therein and binds the polymerized hemoglobin of the hemoglobin solution to produce oxyhemoglobin. As used herein, the term “single flow-through” means that the hemoglobin solution to be oxygenated flows through the filter only once, as opposed to re-circulating the solution through the filter.

Preferably, the filter for the oxygenation methods of the invention is a hydrophobic hollow fiber cartridge. The hydrophobic hollow fiber cartridge refers to a membrane-based oxygenator or gas transfer membrane contactor, known in the art. The hollow fiber cartridges are typically made of hydrophobic polymers, such as polyethylene, polypropylene, or PTFE and are of pore sizes preferably from 0.01 to 0.2 microns. Commercially available hydrophobic hollow fiber membrane contactors include the following: Liqui-Cel mini membrane contactors (G477, Celgard LLC, Division of Membrana, Charlotte, N.C.); FiberFlow hydrophobic capsule filter (SV-C-030-P, Minntech Corporation, Minnetonka, Minn.); and Cell-Pharm Hollow Fiber Oxygenators (Oxy-1, Biovest International, Worcester, Mass.) Technologies. The modules are preferably on the order of 0.5-25 square feet, such as 0.5-5 square feet, 0.5-2.5 square feet or 0.5-1.5 square feet, of membrane area and are composed of materials which can be sterilized by either autoclaving or gamma-irradiation. Preferably, the hemoglobin solution to be oxygenated flows through the hydrophobic hollow fiber cartridge in a different direction than a direction of flow of the oxygen gas, such as in an opposite direction.

The hemoglobin solution to be oxygenated flows through the hydrophobic hollow fiber cartridge at a flow rate preferably in a range of between about 2 mL/minute and about 12 mL/minute, such as between about 4 mL/minute and about 12 mL/minute or between about 10 mL/minute and about 12 mL/minute.

The oxygen gas flows through the hydrophobic hollow fiber cartridge at a flow rate preferably in a range of between about 3 cc/minute and about 25 cc/minute, such as between about 3 cc/minute and about 20 cc/minute or between about 10 cc/minute and about 20 cc/minute.

In a preferred embodiment, when the hemoglobin solution to be oxygenated and an oxygen gas independently flow through a filter, preferably a hydrophobic hollow fiber cartridge, at flow rates as described above, the surface area of the filter is in a range of between about 0.5 ft² and about 25 ft² (or between about 450 cm² and about 2.5 m²), such as between about 0.5 ft² and about 5 ft² (or between about 450 cm² and about 0.5 m²), between about 0.5 ft² and about 2.5 ft² (or between about 450 cm² and about 0.25 m²), between about 0.5 ft² and about 1.5 ft² (or between about 450 cm² and about 1,500 cm²), between about 0.8 ft² and about 1.2 ft² (or between about 700 cm² and about 1,200 cm²), or about 1 ft² (or between about 900 cm² and about 1,000 cm²).

In another preferred embodiment, the hemoglobin solution to be oxygenated flows through a filter, preferably a hydrophobic hollow fiber cartridge, in a single pass-through at an area normalized flow rate in a range of between about 20 mL/min/m² and about 110 mL/min/m² (or between about 2 mL/min/ft² and about 10 mL/min/ft²); and an oxygen gas flows through the filter at an area normalized flow rate in a range of between about 50 cc/min/m₂ and about 300 cc/min/m² (or between about 5 cc/mini ft² and about 25 cc/min/ft²).

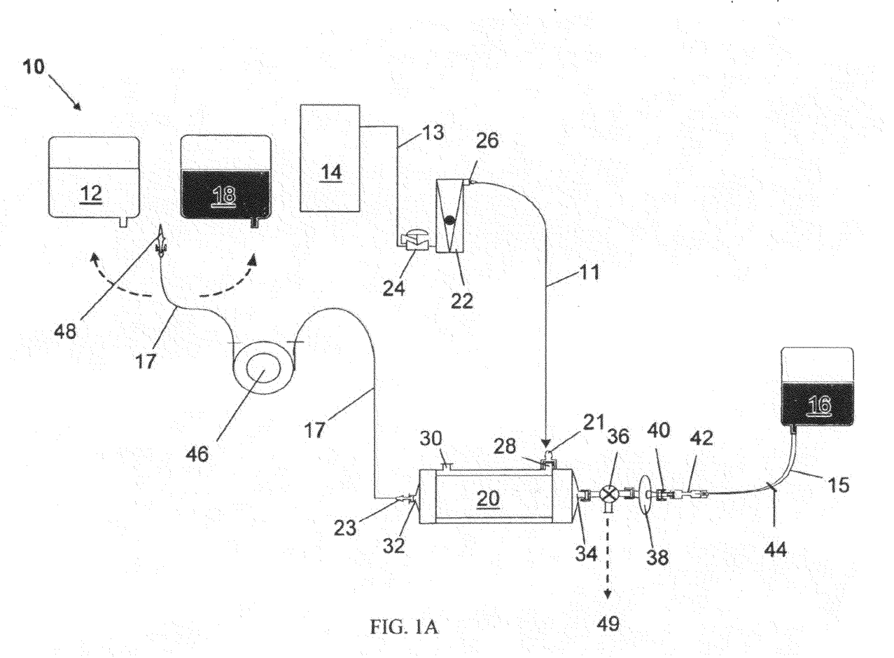

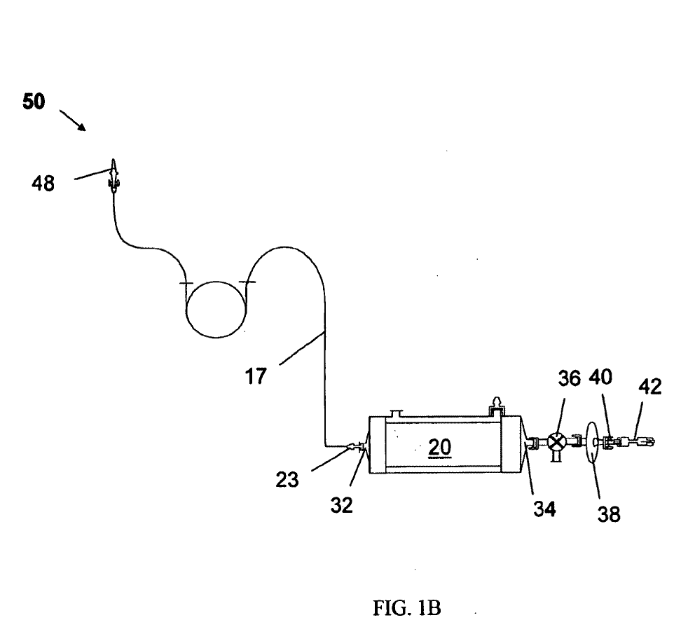

In a specifically preferred embodiment, preparation of an oxygenated hemoglobin solution of the invention is performed using oxygenation system 10 as shown in FIG. 1A. Oxygenation system 10 is a process/apparatus to oxygenate a polymerized hemoglobin solution, such as HEMOPURE® HBOC in vitro. A polymerized hemoglobin solution contained in Hb feed bag 12 is pumped through cartridge 20 (preferably, a hydrophobic hollow fiber cartridge) where a gas exchange occurs. Cartridge 20 allows an oxygen gas to diffuse into the polymerized hemoglobin solution and to bind hemoglobin molecules of the polymerized hemoglobin solution. Preferably, cartridge 20 prevents the polymerized hemoglobin solution from leaking into the gas side of cartridge 20. Typically, cartridge 20 has relatively small-sized pores that can prevent any particles and other contaminations from entering to the polymerized hemoglobin solution through gas inlet 28. The resulting oxygenated hemoglobin solutions, such as oxygenated HEMOPURE® solutions, are collected into pre-sterilized, product collection bag 16 via aseptic connections, such as valve 36, connectors 40 and 42 and filter 38. Depending upon the desired uses, the oxygenated hemoglobin solutions are optionally diluted with USP grade saline supplied from saline supply bag 18 to achieve the desired concentration, such as a concentration where the total amount of hemoglobin is in a range of between about 1.0 and about 25 g/dL, such as between about 1.0 and about 17 g/dL, between about 1.0 and about 14 g/dL or between about 1.2 and about 14 g/dL (e.g., about 6.5-13 g/dL). Oxygen supply to cartridge 20 is controlled by connecting pressurized oxygen gas source 14 (e.g., a bottled medical grade oxygen gas or house oxygen supply) to gas inlet 28 of cartridge 20 through medical grade tubings 11 and 13. An oxygen-gas supply pressure is controlled by pressure regulator 24, and a gas flow is controlled by rotameter 22.

In oxygenation system 10, an oxygen gas enters from oxygen gas source 14 to gas inlet 28 of cartridge 20, contacts hollow fibers of cartridge 20 in an opposite direction to the hemoglobin flow and vents to atmosphere or to a gas collection bag (not shown) through gas outlet port 30 of cartridge 20.

Oxygenation system 10 can allow multiple polymerized hemoglobin solutions to be oxygenated and collected continuously in pre-sterilized product collection bags 16.

In some embodiments, oxygenation system 10 is portable. Optionally, once all of the desired numbers of the oxygenated hemoglobin solutions of the invention have been prepared, the connectors, cartridge and associated tubings are discarded.

In a specifically preferred embodiment, all of the materials necessary for oxygenation system 10, such as tubings, fittings, valves, connectors, cartridge and filters, are sterilized prior to use either by autoclaving at an elevated temperature, such as about 121° C., or by gamma irradiation.

Preferably, oxygen gas source 14, such as a medical grade bottle or facility supply, regulated to a supply pressure of less than about 300 psig, more preferably less than about 100 psig, is attached to the inlet of pressure regulator 24 through tubing 13. Preferably, pressure regulator 24 is adjusted for a feed pressure of between about 5 psig and about 10 psig. Detailed exemplary procedures for setting up and operating oxygenation system 10 are described below:

### A. Preparation of Oxygenation System Components

For assembly 50 shown in FIG. 1B, a pre-assembled, gamma-irradiated assembly can be used. Gamma-irradiation is well known in the art and may be performed by a medical and bioprocess product vendor in the art, such as Charter Medical Ltd, Winston-Salem, N.C. Alternatively, non-sterile components are assembled and steam sterilized in a validated autoclave known in the art. Generally, the autoclaved components should be used within seven days of autoclaving. An exemplary procedure for preparing autoclaved assembly 50 is as follows:

a. Pump 46 (e.g., a peristaltic pump) and tubing 17 are installed as shown in FIG. 1A. For valve 36, a 3-way stopcock is demonstrated herein.

b. waste collection bag 49 is attached to 3-way stopcock 36 via a waste line, and 3-way stopcock 36 is directed to waste collection bag 49.

c. Between about 600 and about 800 mL of USP purified water is placed in a clean depyrogenated glass flask.

d. Tubing 17 is submerged in the USP purified water of the glass flask, and the USP purified water is pumped from the glass flask through assembly 50 and into waste collection bag 49 by operating pump 46 at or greater than about 100 mL/min.

e. After flushing with USP purified water, pump 46 is stopped, the waste line is removed from 3-way stopcock 36, and connector 40 (e.g., a female by female Luer connector) is then connected to 3-way stopcock 36.

f. Assembly 50, including tubing 17, cartridge 20, valve 36, filter 38, connectors 23, 40 and 42, are placed in an autoclave pouch.

g. An autoclave pouch containing assembly 50 is placed in a validated autoclave and autoclaved for about 30-40 minutes.

### B. Oxygenation System 10 Set Up

The process equipment of oxygenation system 10 can be portable and transported to any designated sites. Preferably, the process equipment is set up at a study site in a clean area where aseptic connections can be made.

Oxygen gas source 14 of a medical grade oxygen gas, regulated to a supply pressure of less than about 300 psig, preferably less than about 100 psig, is attached to pressure regulator 24 and rotameter 22. Pressure regulator 24 is adjusted for a feed pressure of about 5-10 psig. Flexible medical grade tubing 11 is connected from the outlet of rotameter 22 to cartridge 20 through connectors 21 and 26, such as barbed Luer fittings.

Product collection bag 16 (e.g., 1000 mL) is connected to aseptic connector 42.

Hb Feed bag 12 containing a polymerized hemoglobin solution (e.g., HEMOPURE® HBOC) or saline supply bag 18 containing USP grade saline is connected to assembly 50 via supply port 48 (e.g., a spike port), for example, by puncturing a spike port of Hb feed bag 12 or saline supply bag 18 with spike port 48.

Prior to the preparation of oxygenated hemoglobin solutions, optionally the assembled components are flushed with saline. Saline is used to prime oxygenation system 10 and can be only required prior to oxygenating the very first bag. One bag of medical (USP) grade saline (e.g., 250 ml) is attached to supply port 48 (e.g., a spike port); one empty waste collection bag 49 (e.g., 1000 ml) is attached to 3-way stopcock 36; and 3-way stopcock 36 is directed towards the attached waste collection bag 49. The pump speed is set at approximately 250 rpm (approximately 75 ml/min) and the entire contents of the saline supply bag are flushed through assembly 50 and collected into the attached waste collection bag 49. Once the saline supply bag has emptied, the pump is stopped and the waste bag is removed and discarded. Subsequently, 3-way stopcock 36 is directed toward product collection bag 16. Oxygenation system 10 is now primed with saline and ready to produce oxygenated hemoglobin solutions, such as oxygenated HEMOPURE® solutions.

### C. Oxygen Flow Procedure

Oxygen gas source 14 has an appropriate pressure (e.g., 10-300 psig, preferably 10-100 psig). A suitable pressure rated hose/tubing is provided for tubings 13 and 11. Oxygen gas source 14 is connected via tubing 13 to gas pressure regulator 24. Tubing 13 and gas pressure regulator 24 are connected with each other by appropriate connectors (e.g., metric or English compression connections). Gas pressure regulator 24 is then adjusted to provide a desired pressure; such as about 5-10 psig of oxygen pressure, to rotameter 22. Subsequently, the rotameter's metering valve is adjusted so that the meter's ball is set at a desired range, for example a between about 10 cc/min and 20 cc/min range. The gas flow setting preferably is checked periodically, e.g., the beginning, during and the end of oxygenation processes.

### D. Polymerized Hemoglobin Oxygenation

The empty saline supply bag used for flushing assembly 50 is removed from supply port 48, and Hb supply bag 12 is connected to supply port 48. Product collection bag 16 is attached to connector 42 through tubing 15, and clamp 44 is opened. Pump 46 is then started, the polymerized hemoglobin of Hb supply bag 12 is oxygenated within cartridge 20, and the resulting oxygenated hemoglobin solution is collected in product collection bag 16.

### E. Saline Dilution

For an optional saline dilution, empty Hb supply bag 12 is removed from supply port 48, and saline supply bag 18 is then connected to supply port 48. Pump 46 is turned on and saline from saline supply bag 18 is transferred to product collection bag 16; diluting the oxygenated hemoglobin solution therein. Once saline supply bag 18 is emptied or once the desired amount of saline is supplied, pump 46 is stopped. Tubing 15 is then clamped and product collection bag 16 is detached from connector 42. The detached product collection bag 16 is then labeled with an approved label and placed on ice or in a refrigerator. Cooling generally maintains a low methemoglobin concentration following the filling at room temperature.

Preferably, the oxygenated hemoglobin solutions of the invention are stored at a temperature of about 15° C. or less. More preferably, the temperature is maintained in a range between about 2° C. and about 8° C.

Although Oxygenation system 10 is illustrated herein to employ one cartridge 20, in some embodiments, more than one cartridge 20 in series or in parallel can be employed. When a plurality of cartridges 20 is employed in parallel, more than one product collecting bag 16 can be employed and connected to each cartridge. More than one oxygen gas source 14 can also be used in these embodiments.

Generally suitable oxygenated hemoglobin solutions employed by the method of the invention are prepared in vitro by oxygenating hemoglobin solutions that include polymerized hemoglobin to convert at least about 80%, more preferably at least about 90%, by weight of the polymerized hemoglobin to oxyhemoglobin. In some embodiments, about 18% by weight, or less, of the polymerized hemoglobin that is included in the hemoglobin solutions to be oxygenated has a Molecular weight of over 500,000 Daltons; about 5% by weight, or less, of the polymerized hemoglobin that is included in the hemoglobin solutions to be oxygenated has a molecular weight equal to or less than 65,000 Daltons; and an endotoxin content of the hemoglobin solution that is included in the hemoglobin solutions to be oxygenated is less than about 0.5 endotoxin units per milliliter, preferably less than about 0.05 endotoxin units per milliliter. Also, a P₅₀ of the polymerized hemoglobin is in a range of between about 24 and about 46 mm Hg, preferably between about 34 and about 46 mm Hg. The oxygenated hemoglobin solutions suitable for use in the method of the invention prepared in vitro can also include one or more pharmaceutically acceptable carriers and/or excipients. Examples of such carriers include water, saline solution, dextrose solution and the like. Examples of excipients include sodium chloride and physiologically-acceptable buffers.

The specifications for a suitable, stable polymerized hemoglobin solution for preparing the oxygenated hemoglobin solutions of the invention are provided in Table 1.

TABLE 1

| Parameter | Results |
|---|---|
| pH (18-22° C.) | Physiologically acceptable |
| Endotoxin | Physiologically acceptable |
| Sterility Test | Meets Test |
| Phospholipids^{a} | Physiologically acceptable |
| Total Hemoglobin | 10-250 g/L |
| Methemoglobin^{b} | <15% |
| Oxyhemoglobin^{b} | <10% |
| Sodium, Na⁺ | Physiologically acceptable |
| Potassium, K⁺ | |
| Chloride, Cl⁻ | |
| Calcium, Ca⁺⁺ | |
| Boron | |
| Glutaraldehyde | Physiologically acceptable |
| N-acetyl-L-cysteine | Physiologically Acceptable |
| M.W. > 500,000 | 18% |
| M.W. < 32,000 | <5% |
| Particulate Content > 10μ | <12/ml |
| Particulate Content > 25μ | <2/ml |

^{a}measured in Hb before polymerization.
^{b}based on the total hemoglobin.

Plegic and cardioplegic solutions are known to those skilled in the art. Plegic solutions are solutions that mimic certain physiological properties of blood or plasma and are used to stabilize and preserve the viability of organs or tissues in vivo or ex vivo for a limited period of time. Cardioplegic solutions are plegic solutions that are used specifically to stabilize and preserve viability of the heart or tissues removed from the heart. Generally, two types of cardioplegic solutions are used: 1) amino acid-enriched solutions or blood cardioplegia that normally contain monosodium glutamate (MSG) and monosodium aspartate (MSA), CPD, Dextrose, Thromethamine and KCl; and 2) crystalloid solutions that do not contain MSG/MSA.

In one embodiment, the invention employs an oxygenated hemoglobin solution that includes from about 10 grams to about 250 grams of polymerized hemoglobin per liter of solution. In the oxygenated hemoglobin solution: a) about 80% by weight, or greater, of the polymerized hemoglobin of the oxygenated hemoglobin solution is oxyhemoglobin; b) about 18% by weight, or less, of the polymerized hemoglobin has a molecular weight of over 500,000 Daltons; c) about 5% by Weight; of less, of the polymerized hemoglobin has a molecular weight equal to or less than 65,000 Daltons; d) a P₅₀ of the polymerized hemoglobin is in a range of between about 34 and about 46 mm Hg; and e) an endotoxin content of the oxygenated hemoglobin solution is less than about 0.05 endotoxin units per milliliter.

The term “P₅₀” is recognized in the art as a term employed to describe the interaction between oxygen gas (O₂) and hemoglobin, and represents the partial pressure of oxygen gas (pO₂) at 50% saturation of hemoglobin. Thus, “a P₅₀ of polymerized hemoglobin” indicates interaction between oxygen gas (O₂) and the polymerized hemoglobin. This interaction is frequently represented as an oxygen dissociation curve with the percent saturation of hemoglobin plotted on the ordinate axis and the partial pressure of oxygen in millimeters of mercury (mm Hg) or torrs plotted on the abcissa. Preferably, a P₅₀ of the polymerized hemoglobin that can be employed in the invention is in a range of between about 24 mm Hg and about 46 mm Hg, more preferably between about 34 mm Hg and about 46 mm Hg. In one embodiment, the P₅₀ of the polymerized hemoglobin is about 40 mm Hg.

In one embodiment, the oxygenated hemoglobin solution has a viscosity of between about 1 centipoise and about 2 centipoise at about 37° C. In another embodiment, the oxygenated hemoglobin solution has a viscosity of between about 1 centipoise and about 1.5 centipoise at about 37° C. In another embodiment, the oxygenated hemoglobin solution has a viscosity of about 1.3 centipoise at about 37° C. In another embodiment, the oxygenated hemoglobin solution has a viscosity of between about 1 centipoise and about 2 centipoise at about 37° C. and wherein a P₅₀ of the polymerized hemoglobin is in a range of between about 34 and about 46 mm Hg.

In a one embodiment, the oxygenated hemoglobin solution is administered to the subject at a temperature in a range of between about 18° C. and about 37° C. In another embodiment, the oxygenated hemoglobin solution is administered to the subject at a temperature in a range of between about 25° C. and about 37° C. In a specific embodiment, the oxygenated hemoglobin solution is administered to the subject at a temperature of about 37° C.

In a one embodiment, the oxygenated hemoglobin solution is administered by infusion. Delivery of the oxygenated hemoglobin may be by intra-arterial infusion, or via retrograde infusion into the venous circulation of an ischemic organ or into the central venous circulation of an organism.

In a one embodiment, the oxygenated hemoglobin solution is infused at a rate in a range of between about 10 ml/minute and about 200 ml/minute. In another embodiment, the oxygenated hemoglobin solution is infused at a rate in a range of between about 20 ml/minute and about 100 ml/minute. In another embodiment, the infusion rate is in a range of between about 40 ml/minute and about 60 ml/minute. In a specific embodiment, the infusion rate is in a range of about 48 ml/minute.

In a one embodiment, the oxygenated hemoglobin solution further includes one or more physiological ions. In one embodiment, the physiological ions include potassium, sodium and chloride ions. In another embodiment, the physiological ions include potassium, sodium, chloride and calcium ions.

In another embodiment, the oxygenated hemoglobin solution further includes glucose where the concentration of glucose is between about 0 and about 50 millimoles per liter. In a specific embodiment, the glucose concentration is about 11 millmoles per liter. In another embodiment, the oxygenated hemoglobin solution further includes insulin wherein the concentration of insulin is between about 0 and about 1,000 milliunits per liter. In a specific embodiment, the insulin concentration may be about 50 milliunits per liter. In another embodiment, the concentration of potassium concentration in oxygenated hemoglobin solution is about 0 to about 100 millimoles per liter. In an specific embodiment, the potassium concentration may be about 4.5 millimoles per liter. In an alternative embodiment, the solution includes a physiological buffer which includes at least one component selected from the group consisting of: sodium lactate, N-acetyl-L-cysteine, sodium chloride, potassium chloride, and calcium chloride.2H₂O. In an specific embodiment, the concentration of sodium lactate is about 0 to about 45 millimoles per liter. In another specific embodiment, the concentration of N-acetyl-L-cysteine is about 0 to about 0.2%. In another specific embodiment, the concentration of sodium chloride is about 145 to about 160 millimoles per liter. In yet another specific embodiment, the concentration of potassium chloride is about 0 to about 100 millimoles per liter. In another specific embodiment, the concentration of calcium chloride.2H₂O is about 0.5 to about 1.5 millimoles.

In one embodiment, the patient has acute ischemia or acute angina. In one embodiment, the ischemia or angina is caused by an arterial intervention or surgery. In another embodiment, the ischemia or angina is caused by a cardiac surgery. In another embodiment, the ischemia results in a myocardial infarction of which angina may be one symptom. Clinical indications for the use of this invention include, but are not limited to the following procedures or interventions during which ischemia will be prevented by intra- or peri-procedural infusion the disclosed oxygenated hemoglobin solutions: angioplasty, arterial stent deployment, angiography, angioscopy, atherectomy, bypass grafting including coronary (CABG) and peripheral bypass grafting, endarterectomy, organ transplantation, cardiopulmonary bypass surgery, embolectomy, thrombolytic therapy, aortic surgery (especially that which interrupts blood flow to the brain, liver or kidneys) and mesenteric tissue revascularization.

In another embodiment, the tissues include brain, lung, liver, pancreas, spleen, kidney, heart, sections of small intestine, sections of large intestine, rectum, pancreas, skeletal muscles, stomach, urinary bladder, esophagus, larynx, trachea, bronchi, glands including sublingual, parotid submaximal and thyroid glands.

In another embodiment, the vessels include the following arteries: internal carotid, right vertebral, brachiocephalic, sublavian, axillary, dep brachial, brachial, common hepatic, hepatic proper, gastroduodenal, right gastric, right gastroepiploic, superior mesenteric, middle colic, right colic, ileocolic, radial, ulnar, deep palmar arch, superfacial palmer arch, digital, common iliac, internal iliac, external iliac, dorsalis pedis, arcuate, metatarsals, ophthalmic, maximallary, facial, lingual, external carotid, right common carotid, aortic arch, thoracic aorta, abdominal aorta, diaphragm, inferior phrenic celiac trunk, splenic, left gastric, left gastroepiploic, suprarenal, renal, gonadal, left colic, inferior mesenteric, sigmoidal, superior rectal; medial sacral, femoral, popliteal, anterior tibial, posterior tibial, peroneal, plantar arch, and digital. In addition, the veins of the heart, lung, liver, kidneys, spleen, pancreas, skeletal muscles, urinary bladder, glands (including sublingual, parotid submaximal and thyroid glands) may be retrogradely perfused with oxygenated hemoglobin glutamer-250 (bovine), hemoglobin-based oxygen carrier.

In one embodiment, the oxygenated hemoglobin solution further includes an additional therapeutic agent. Additional therapeutic agents include oxfenicine (about 0 to about 100 mmol/l), N⁶-cyclohexyladenosine (about 0 to about 100 μmol/l), mannitol, magnesium, procaine, bicarbonate, tromethamine, mono-sodium L-glutamate monohydrate and monosodium L-aspartate monohydrate, dextrose, glacial acetic acid, citrate, phosphate, dextrose, monobasic sodium phosphate, albumin, sorbitol and aspartate.

In a one embodiment, the oxygenated hemoglobin solution is a glutaraldehyde-polymerized hemoglobin solution from isolated bovine red blood cells. In another embodiment, the oxygenated hemoglobin solution is oxygenated hemoglobin glutamer-250 (bovine), hemoglobin-based oxygen carrier that includes a hemoglobin concentration about 6.5-about 13.0 g/dL, greater than about 90 wt % oxyhemoglobin, less than about 5 wt % methemoglobin. An endotoxin content of oxygenated HEMOPURE® HBOC is less than about 0.05 endotoxin units per milliliter. In oxygenated hemoglobin glutamer-250 (bovine), hemoglobin-based oxygen carrier, about 18% by weight, or less, of the polymerized hemoglobin has a molecular weight of over about 500,000 Daltons, and about 5% by weight, or less, of the polymerized hemoglobin has a molecular weight equal to, or less than about 65,000 Daltons.

For intracoronary indications described above, the administration of oxygenated HEMOPURE® HBOC has been optimized via an empirically-determined selection of conditions that include catheter style (Helios balloon catheter), infusate temperature (37° C.) and infusion rate (48 ml/min in an adult human). Intracoronary administrations of other agents (e.g., contrast agents, drugs, saline) are typically delivered at room temperature. The viscosity of oxygenated HEMOPURE® HBOC decreases with increasing temperature between 4° and 37° C. Delivering oxygenated HEMOPURE® HBOC at 37° C. delivers this therapeutic at the lowest possible viscosity which, in turn, maintains a lower intracatheter pressure and low infusate jet action at the distal catheter tip. Minimizing intracoronary jet action protects coronary endothelial integrity. Infusing at 37° C. also optimizes preservation of cardiac function during coronary occlusion. These collective conditions accomplish the simultaneous goals of delivering sufficient oxygen to meet myocardial oxygen demand at a temperature that maximizes preservation of myocardial function, with minimum perturbation of the coronary endothelium.

### EXEMPLIFICATION

### Example 1

### Oxygenation of Polymerized Hemoglobin Solutions

HEMOPURE® HBOC as described in Table 1, was oxygenated by a method as described above, using oxygenation system 10. In this example, for each 1000 mL product collection bag 16, one 250 mL saline supply bag 18 and one Hb supply bag 12 containing HEMOPURE® HBOC were used.

Specific components used for oxygenation system 10 for this example are summarized in Table 2 below:

TABLE 2
System Equipment

| Description | Specifications |
|---|---|
| Minntech Fiberflo Hollow | 0.03 μm pore size, 1 ft² membrane area |
| Fiber Capsule (SV-C-030-P) | |
| for cartridge 20 | |
| Watson-Marlow 323E/D | 0−>100 mL/min flow range, PLC |
| peristaltic pump for pump 46 | controllable |
| Bioprene autoclavable tubing | 4.8 OD × 1.6 mm ID |
| for tubing 17 | |
| Tygon tubing for tubings 11 | 5/32 ID × 7/32 OD |
| and 13 | |
| Male and female Luer Lock × | Polypropylene |
| 1/16 hose barb for | |
| connectors 21 and 23 | |
| Cole Parmer rotameter | 0-60 cc/min range |
| (PMR1-011487) for | |
| rotameter 22 | |
| Watts Fluidair Pressure | 0-300 psig inlet pressure, 0-60 psig |
| Regulator (R364-01AG) for | adjustable outlet pressure |
| pressure regulator 24 | |
| CharterBio BP100BSB bag | Volume 1000 mL |
| assembly w/Needle-less | |
| injection site for connectors | |
| 40 and 42 | |
| Pall 32 mm sterilizing filter | Supor 0.2 μm membrane, Female Luer |
| for filter 38 | Lock inlet, Male Luer Lock outlet |

A medical grade oxygen gas was used and the oxygen gas concentration of oxygen source 14 was greater than 99%. Pressurized oxygen supply was regulated to less than 100 psi, and pressure regulator 24 was rated to a 100 psig inlet pressure. The polymerized hemoglobin solution flow rate was 10-12 mL/min. The oxygen gas flow rate was 10-20 cc/min. Resulting product collection bag 16 in this example contained an oxygenated hemoglobin solution in which a hemoglobin concentration was approximately 6.5±approximately 1.0 g/dL and an oxyhemoglobin content was greater than approximately 90%, as summarized in Table 3 below:

TABLE 3
Specifications of Oxygenated HEMOPURE ® HBOC

| | Specifications (wt %) |
|---|---|
| Oxy Hemoglobin^{a} | >90% |
| Met Hemoglobin^{a} | <5% |
| total Hemoglobin | 6.5 ± 1.0-13 ± 1.5 g/dL |

^{a}based on the total hemoglobin.

### Example 2

### Physiochemical and Biochemical Properties of HEMOPURE® HBOC

HEMOPURE® HBOC (see Table 1) is a cell-free, endotoxin free, glutaraldehyde-polymerized hemoglobin solution extracted from isolated bovine red blood cells (see Horn, E P. Proceedings of the ASA Congress. 1999; Horn E P, et al., Surgery. 1997; 121:411-418; and Standl T, et al. Can J. Anaesth. 1996; 43:714-723, the entire teachings all of which are incorporated herein by reference), that was initially developed as an alternative to red blood cell transfusions for anemic surgical patients.

By facilitating diffusive oxygen delivery (oxygen diffusion) and convective oxygen delivery (oxygen transport), HEMOPURE® HBOC may act as a direct oxygen donor and “oxygen bridge” between red blood cells and tissues. HEMOPURE® HBOC is characterized by an oxygen equilibrium curve that is right-shifted compared to that of native human hemoglobin, resulting in a P₅₀ (the partial pressure of oxygen at which the Hb is 50% saturated) of approximately 40 mm Hg. This property facilitates oxygen off-loading to tissues. In addition, the viscosity of oxygenated HEMOPURE® HBOC is 1.3 centipoise at 37° C., significantly lower than that of human blood (4 centipoise)(see Rentko V T, Pearce L B, Moon-Massat P F, Gawryl M S. Hemopure (HBOC-201, Hemoglobin Glutamer-250 (Bovine)): Preclinical studies. Pages 424-436. In: R M Winslow, Editor, Academic Press, London, 2006, the entire teachings of all of which are incorporated herein by reference). The comparatively small size (vs. RBCs) and low viscosity of oxygenated HEMOPURE® HBOC relative to blood enables oxygenated HEMOPURE® HBOC to access remote spaces in tissues not accessible to RBCs and to function as an oxygen “bridge” between RBCs and the endothelium, effectively shuttling oxygen to tissues. As a likely manifestation of these properties, administration of oxygenated HEMOPURE® HBOC to dogs following hemodilution with hydroxyethyl starch augmented liver oxygen tension above that induced by hemodilution followed by lactated Ringer's solution (see Freitag M, Standl T G, Gottschalk A, Burmeister M A, Rempf C, Horn E P, Strate T, Schulte am Esch J. Enhanced central organ oxygenation after application of bovine cell-free hemoglobin HBOC-201. Can J Anesth, 52: 904-914, 2005, the entire teachings of all of which are incorporated herein by reference). Exchange transfusion with ultra-purified polymerized bovine Hb solution resulted in a more homogeneous tissue pO₂ distribution, consistent with improved tissue oxygenation (see Botzlar A, Nolte D, Messmer K. Effects of ultra-purified polymerized bovine hemoglobin on the microcirculation of striated skin muscle in the hamster. Eur J Med. Res. 1996; 1:471-478, the entire teachings of all of which are incorporated herein by reference).

The salutary effect of intra-arterial oxygenated HEMOPURE® HBOC is temperature-dependent and is greater when infused at 37° C. than when infused at temperatures below 37° C. The salutary effect of oxygenated HEMOPURE® HBOC is dose-dependent. When infused into coronary arteries, the optimum infusion rate is approximately 50% higher than baseline blood flow to the affected tissue. The lower viscosity of oxygenated HEMOPURE® HBOC, particularly at 37° C., allows for coronary infusion at a higher elevated Hb concentration than would likely be possible with dilute blood. Oxygenated HEMOPURE® HBOC displaces red blood cells in vitro and in vivo and ex vivo by a HEMOPURE® HBOC for the purpose of imaging coronary arteries via OCT.

### Example 3

### In Vitro Results

In all studies described, HEMOPURE® HBOC was oxygenated using a device specially designed and validated for this purpose as described in Example 1.

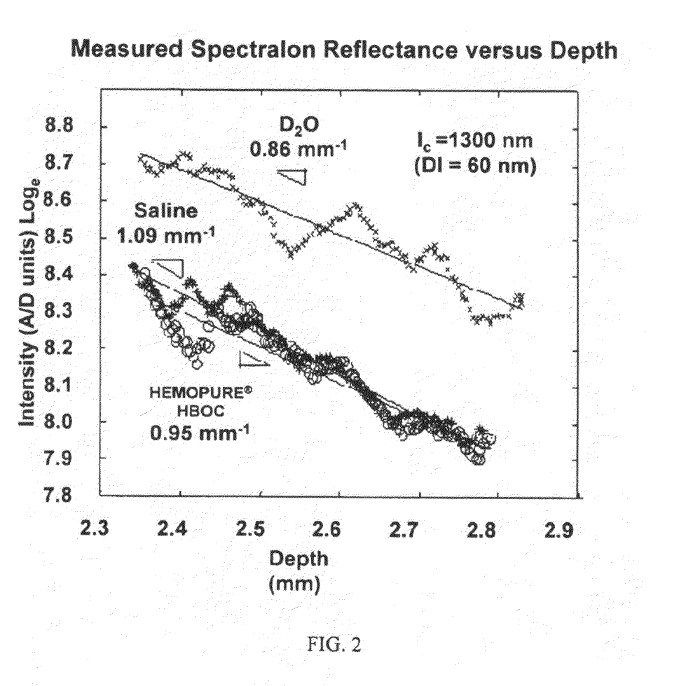

We have characterized in vitro that oxygenated HEMOPURE® HBOC transmits near-infrared light (1310 nm) efficiently with an attenuation that is similar to that of saline (<0.15 mm⁻¹) (FIG. 2). The refractive index of oxygenated HEMOPURE® HBOC is 1.357.

We have also demonstrated in vitro that oxygenated HEMOPURE® HBOC can be infused through the lumen of an angioplasty catheter (Maverick model, Boston Scientific, Natick, Mass.) and an OCT imaging catheter (Helios Short-nose imaging catheter, Light Labs Imaging, Inc., Westford, Mass.) using a typical clinical syringe pump (MedRad V Pro-Vis) at infusion rates up to 60 ml/min, consistent with rates appropriate for intracoronary infusion in pigs and humans. We have also shown that it is possible to collect high-quality images of artery architecture when oxygenated HEMOPURE® HBOC is infused via an OCT imaging catheter. This characterization included documentation of the relationships between intracatheter pressures, infusion rates, infusate temperature and jetting action as the infusate exits the catheter tip.

We have also generated data that characterizes in vitro, the process for warming oxygenated HEMOPURE® HBOC during infusion using a commercially available clinical fluid warmer Astotherm Plus® (Futuremed America, Inc. Granada Hills, Calif.).

### Example 4

### In Vivo Results: COR-0002: Evaluation of HBOC Therapeutics in Elective Percutaneous Coronary Revascularization

The safety and tolerability of HBOC in cardiac patients scheduled for elective PCI has recently been investigated in the COR-0001 trial. (See Serruys P W, Vranckx P, Slagboom T, Regar E, Meliga E, de Winter R. J, Heyndrickx G, van Remortel E A M, Dubé GP and Symons J for the COR-0001 trial investigators. Hemodynamic effects, safety, and tolerability of hemoglobin-based oxygen carrier-201 (HBOC-201) in patients undergoing PCI for CAD. EuroIntervention Journal. 3: 600-609, 2008, the entire teachings of all of which are incorporated herein by reference). This study showed that IV HBOC administration maintained the function of the heart but did not compromise autoregulation of coronary blood flow, despite the known vasoconstrictive properties of this drug, or myocardial function as assessed by the left ventricular stroke work. A transient increase in the mean arterial blood pressure (MAP) and systemic vascular resistance was observed, consistent with the purported nitric-oxide scavenging activity of the drug.

### Methods

### Study Design

The COR-0002 pilot trial was a single-center, phase II, placebo-controlled, cross-over, single-blind study conceived to test the hypothesis that HBOC administration improves myocardial “oxygenation” and myocardial function during brief coronary occlusion. Enrolled subjects underwent coronary balloon occlusion, with and without oxygenated HBOC intracoronary infusion (11-13 g/dl at 48 ml/min up to 3 min).

### Study Procedures:

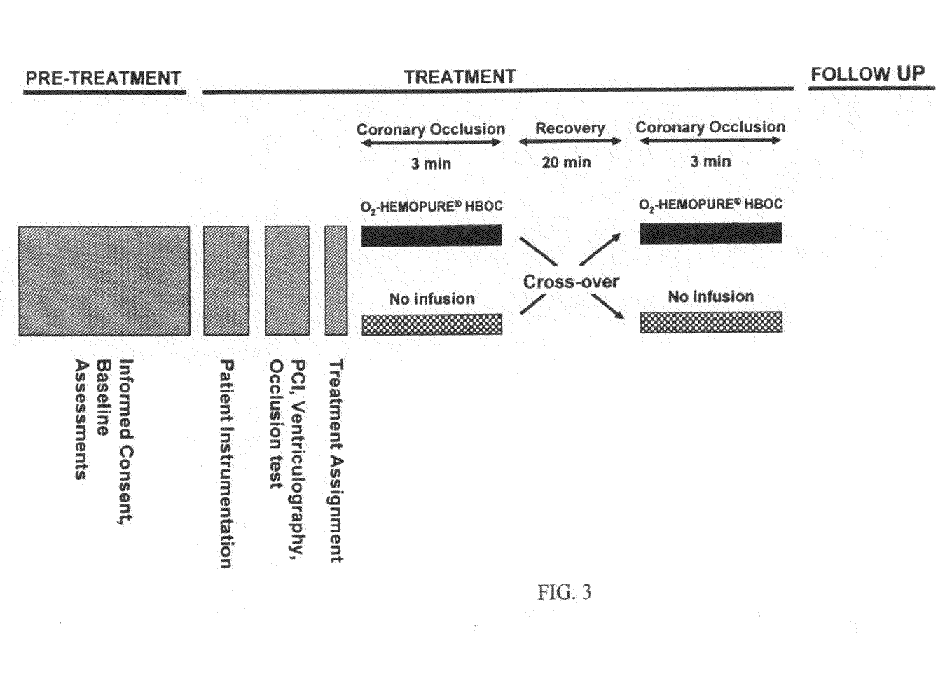

Data collection points and study design are depicted in FIG. 3. All subjects were followed from study inclusion to hospital discharge.

### Continuous 12-Lead Holter Monitoring

On arrival in the cath lab, the patient was connected to a continuous 12-lead Holter ECG recording device. ST-segment shift compared to baseline was analyzed in the lead that demonstrated the most severe alterations as well as in all leads showing ST-segment changes≧1 mm (at 60 ms after the J-point).

### Patient Instrumentation.

Vascular access was obtained using the femoral approach with a standard Seldinger technique. Usually, a 6 or 7 French arterial sheath was selected. Prior to starting the PCI procedure, a conductance catheter was inserted into the left ventricle by an additional 8 french arterial sheath (details concerning the hemodynamic data acquisition are provided below in “LEFT VENTRICULAR HEMODYNAMICS”). A Swan-Ganz catheter was placed in the pulmonary artery via the femoral vein for cardiac output determinations by thermal and hypertonic saline (NaCl 10%) dilution methods.

### Index PCI Procedure Phase

An index PCI procedure was performed according to standard institutional practices.

### Study Phase

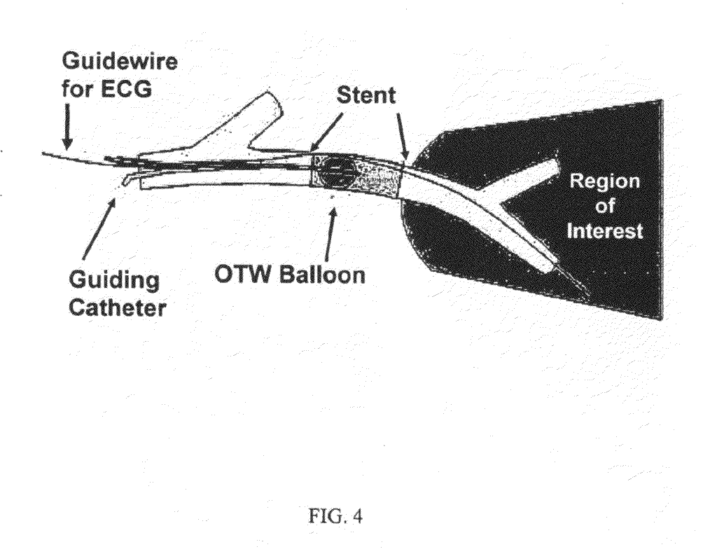

A short over-the-wire (OTW) balloon (Helios 1.5, Goodman, Japan) was positioned inside the stent using a conventional 0.014 inch guide wire (Balance Middle Weight, Guidant). A conventional 0.014-inch guide wire (Balance Middle Weight, Guidant) was inserted outside the OTW balloon, distal to the stent (in the “region of interest”) to allow for continuous “online” intracoronary ECG monitoring (FIG. 4).

### Study Occlusion and Fluid Infusion

All subjects underwent two intra-stent balloon occlusions (balloon inflation pressure=0.5 atm). During one occlusion, a continuous intracoronary infusion of pre-oxygenated HEMOPURE® HBOC, warmed to 37° C., was administered through the OTW lumen at a rate of 48 ml/min (maximum volume infused is 144 ml). HEMOPURE® HBOC was warmed via an in-line clinical fluid warmer (Astotherm® plus, Model AP220S, Futuremed America, Inc., Granada Hills, Calif., USA) positioned immediately proximal to the intracoronary OTW helios balloon catheter. HEMOPURE® HBOC was contained within the sterile, high-pressure infusion line wrapped around the heating coil of the clinical fluid warmer.

The control occlusion period was performed similarly, but without infusion (termed “dry occlusion”). Subjects were assigned to receive pre-oxygenated HEMOPURE® HBOC during the first occlusion period and no-infusion during the second period or vice versa. Each occlusion and infusion period lasted for up to 3 minutes. Three patients received oxygenated HEMOPURE® HBOC during the first coronary occlusion and two patients received a dry occlusion as the first experimental intervention. Pre-determined criteria for premature interruption of the balloon occlusions were:
≧100% increase of left ventricular end-diastolic pressure (LVEDP) from baseline
Sustained ventricular arrhythmias (ventricular tachycardia or ventricular fibrillation)
Intolerable chest pain (angina)
Significant hypertension (systemic blood pressure rise to >180 mmHg)
Significant LV dysfunction (Ejection fraction (EF) decrease to less than 35%)

Once the balloon had been deflated and the infusion stopped, a “resting period” of 20 minutes followed for all recorded parameters to return to baseline (in particular LVEDP). The treatment period concluded after, the second deflation, once all parameters had returned to their baseline values.

### Left Ventricular Hemodynamics

Left ventricular hemodynamic data were recorded before, during and after the procedure by online left ventricular pressure-volume signals obtained by a 7 F combined pressure-conductance catheter (CD Leycom, Zoetermeer, the Netherlands) introduced into the left ventricle via the femoral artery. The catheter was connected to a Cardiac Function Lab (CFL-512, CDLeycom) for display and acquisition of pressure-volume loops. Parallel conductance and cardiac output were determined by multiple injections of hypertonic saline solution and thermodilution, respectively, in order to calibrate the volume signals of the conductance catheter. Data analysis was performed off-line by custom-made software. Cardiac function was quantified by cardiac output and stroke volume, stroke work, end-diastolic and end-systolic volume, LV ejection fraction, end-systolic and end-diastolic pressure, maximal and minimal rate of LV pressure change (dP/dt_{MAX} and dP/dt_{MIN}). The isovolumic relaxation period (defined as the period between the time point of dP/dt_{MIN} and the time point where dP/dT reached 10% of the dP/dt_{MAX} value) was analyzed using phase-plot analysis and the time constant of relaxation (Tau) was then determined. Systemic hemodynamics were quantified by systolic, diastolic and mean systemic arterial pressure recorded every 3 minutes through the guiding catheter for the duration of the study period to investigate any possible hypertensive effects of HEMOPURE® HBOC infusion.

### Efficacy Endpoints

The primary endpoints of this study were early signs of myocardial ischemia during intra-stent balloon inflation defined as changes in left ventricular relaxation (Tau and dP/dT_{MIN}) and changes in the sum of ST segment deviations (assessed by continuous 12-lead Holter ECG monitoring) compared to baseline.

Secondary endpoints included changes in the cardiac performance measured by LV pressure volume loop analysis, clinical signs of myocardial ischemia and changes in coronary vascular tone measured by QCA.

### Statistical Analysis

Variables with normal distribution were analyzed using parametric tests while variables with a non-normal distribution were analyzed with non-parametric tests. Continuous variables are expressed as mean±SD or median±inter-quartile range (IQR) and differences were compared using Student t test or Mann Whitney test. Categorical variables are expressed as counts and percentages.

All values were normalized in order to account for baseline variability. Normalization was done by dividing each response to treatment (HBOC-infusion or dry occlusion) by the respective baseline. This method of normalization was selected because it was an appropriate strategy to minimize the variability associated with differences in baselines between subjects. Differences were assessed by T-test or chi-square test. All statistical tests were two-tailed. A P value <0.05 was considered significant. Due to the descriptive nature of this study, no sample estimation was utilized.

### Results

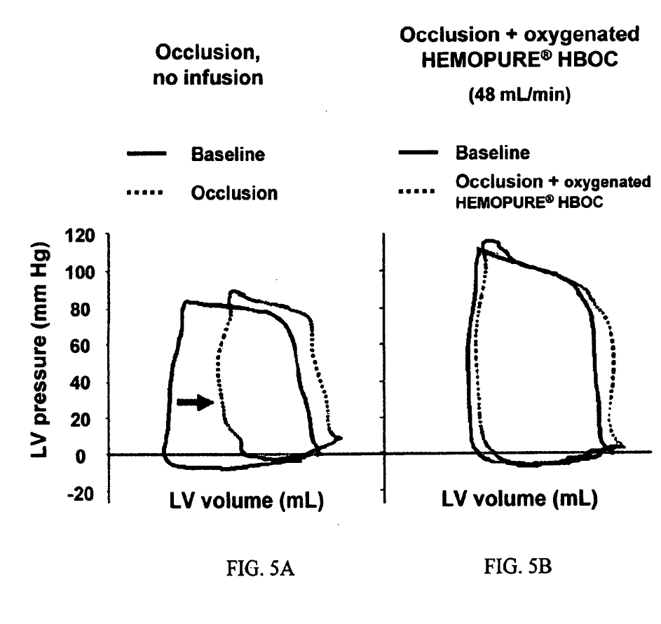

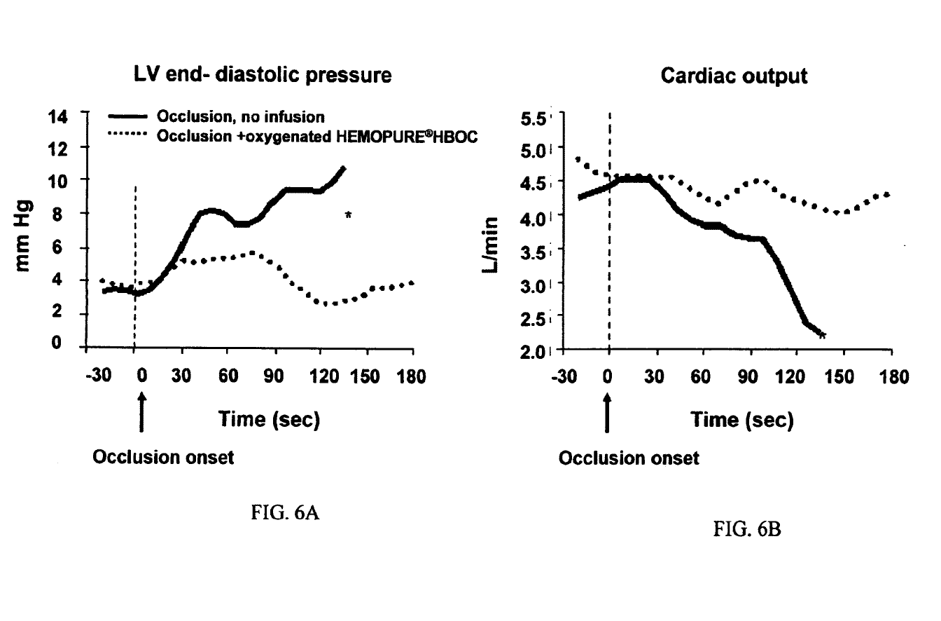

Patients (n=5) of mean age of 54.4±14 years underwent stent implantation for proximal (n=5) and/or mid LAD lesion (n=1) and were enrolled in the present study. The mean ejection fraction (EF) was 66±10%. Procedural and hemodynamic results are illustrated in FIGS. 5A, 5B, 6A and 6B. None of the measured haemodynamic parameters differed significantly from their respective baseline values during HBOC infusion. When data obtained during HBOC infusion and dry occlusion phases were compared, a statistical difference was shown for all systolic and diastolic performance indexes evaluated except dP/dT_{MAX}. EF, CO and dP/dT_{MIN} decreased significantly during dry occlusions from median values of 0.98 (IQR 0.09), 1 (0.06) and 0.99 (0.08) to 0.78 (0.22), 0.9 (0.16) and 0.85 (0.16), respectively. EDP and Tau increased significantly during dry occlusions from median values of 1 (0.39) and 1 (0.16) to 1.5 (0.71) and 1.21 (0.23), respectively. During dry occlusion, an important rightward shift of the PV loop occurred in all patients except PT005, while during HBOC infusion, ESV and EDV did not increase with the exception of PT004.

Intra-stent occlusions performed with infusion of pre-oxygenated HBOC-210 all lasted the intended 3-minute duration; specifically, criteria for premature interruption of the inflation were never met. However, mean duration for dry occlusions was 2.13±0.12 min and premature termination of the occlusion was necessary in all subjects. Table 4 identifies the reason(s) for terminating the dry occlusion in each patient.

TABLE 4
Reasons for premature interruption of dry occlusion phase

| | Premature | Time | | |
|---|---|---|---|---|
| | (Y/N) | (min) | Main reason | Additional reason |
| PT001 | Y | 2.15 | LVEF < 35% | multiple extrasystoles |
| PT002 | Y | 2.05 | EDP > 20 | multiple extrasystoles |
| | | | mmHg | |
| PT003 | Y | 2.46 | LVEF < 35% | |
| PT004 | Y | 2.00 | VT | |
| PT005 | Y | 2.31 | Chest pain | multiple extrasystoles |

LVEF, left ventricular ejection fraction, EDP, end-diastolic pressure, VT, ventricular tachycardia.

During HBOC infusion, ST segment showed no significant changes from baseline while it was found to be significantly elevated during the dry occlusion phase in patients 3, 4 and 5.

From a safety point of view, mean systolic blood pressure (SBP) increase during the HBOC infusion was 10.8±10.5 mmHg. SBP never reached the critical values defined prospectively for protocol-specified pharmacological intervention with nitrates and/or nifedipine.

No noteworthy findings in the clinical chemistry parameters and no serious adverse events in any patient through the 4-day follow-up phase occurred.

### Discussion

The main results of this study are: 1) Intracoronary infusion of oxygenated HEMOPURE® HBOC maintained left ventricular haemodynamic status during total proximal LAD occlusion; 2) LV systolic and diastolic properties were not affected during coronary occlusion with HEMOPURE® HBOC infusion while they significantly deteriorated during the dry occlusion; 3) intracoronary ECG showed no significant ST segment changes during HBOC infusion; 4) HEMOPURE® HBOC did not cause any adverse event or significantly alter blood chemistry parameters through the follow-up period.

The COR-0002 study was designed to test the hypothesis that pre-oxygenated HEMOPURE® HBOC is capable of supporting myocardial metabolism and preserving function during total coronary occlusion in humans. The experimental design selected is a sequential intra-stent angioplasty balloon inflation model with intracoronary infusion of pre-oxygenated HEMOPURE® HBOC compared to the same occlusion with no infusion. Parameters of systolic and diastolic function and ST segment changes were measured to determine whether intra-coronary delivery of oxygenated HEMOPURE® HBOC to myocardium at risk mitigates ischemia.

The evaluation of early signs of myocardial ischemia in this trial relied on continuous, invasive recording of PV loops, a technique able to provide detailed, reliable data on ventricular and myocardial performance throughout the entire cardiac cycle (see Burkhoff D, Mirsky I, Suga H. Assessment of systolic and diastolic ventricular properties via pressure-volume analysis: a guide for clinical, translational, and basic researchers. Am J Physiol Heart Circ Physiol 2005; 289(2):H501-12, the entire teachings of all of which are incorporated herein by reference). Isovolumic relaxation was evaluated by the peak rate of pressure decline (dP/dT_{MIN}) and by the ventricular relaxation time constant Tau (τ). During the dry occlusion phase, early after balloon inflation, dP/dT_{MIN} significantly decreased while τ significantly increased, both early indicators of myocardial ischemia. During HBOC infusion, neither dP/dT nor Tau changed significantly from baseline-values, suggesting HBOC substantially mollified the ischemia otherwise induced by balloon inflation. Alterations of the isovolumic relaxation phase are the earliest and most sensitive signs of ischemia-induced left ventricular dysfunction. In this series of subjects, LVEDP did not significantly increase during HBOC infusion, but increased continually in all subjects during the dry occlusion.

Akin to diastolic function, systolic functions are importantly influenced by ischemia. HBOC largely averted systolic dysfunction. Ejection fraction and stroke volume (SV), major indexes of the ejection phase properties, did not show significant variations from baseline during coronary occlusions with HBOC infusion while they were significantly reduced during the dry occlusion phase. These results were achievable despite the fact that the occlusions were performed in the proximal part of the LAD coronary artery which supplies a large “area at risk.” Consistent with these results, dP/dT_{MAX}, commonly used as an isovolumic phase index of cardiac contractility, did not vary significantly from baseline during HBOC infusion suggesting preservation of systolic myocardial performance.

Early electrical signs of ischemia in the area of interest were detected by using intracoronary ECG, a technique able to detect early signs of ischemia with high sensitivity (see Friedman P L, Shook T L, Kirshenbaum J M, Selwyn A P, Ganz P. Value of the intracoronary electrocardiogram to monitor myocardial ischemia during percutaneous transluminal coronary angioplasty Circulation 1986; 74; 330-339, the entire teachings of all of which are incorporated herein by reference). In this series of patients, HBOC infusion protected against alteration of the ST segment during coronary occlusion. On the contrary, a significant ST elevation, a sign of transmural ischemia, was detected in 3 out of 4 patients during dry occlusion phase. In PT001, the ST elevation did not reach significant levels most likely because the occlusion was interrupted relatively early, due to severe EF impairment (EF<35%) and the presence of multiple extrasystolic beats. In all patients, ST tended to remain elevated even after the balloon deflation while no ST alterations occurred during the resting periods following HBOC infusion.

In summary, intracoronary oxygenated HEMOPURE® HBOC represents a new category of pharmacologic strategies that may have utility in patients undergoing PCI. The results of this exploratory trial provide preliminary evidence that HEMOPURE® HBOC can effectively preserve myocardial mechanical and electrical properties in the face of total coronary occlusion.

### EQUIVALENTS

While this invention has been particularly shown and described with references to example embodiments thereof, it will be understood by those skilled in the art that various changes in form and details may be made therein without departing from the scope of the invention encompassed by the appended claims.

What is claimed is:

1. A method of delivering oxygen to a tissue, a blood vessel, an organ, a region of an organ or an organism under an ischemic condition of a subject, comprising the step of administering to the subject an oxygenated hemoglobin solution, wherein the oxygenated hemoglobin solution includes polymerized hemoglobin, and wherein about 80% by weight, or greater, of the polymerized hemoglobin is oxyhemoglobin.

2. The oxygenated hemoglobin solution of claim 1, wherein about 90% by weight, or greater, of the polymerized hemoglobin is oxyhemoglobin.

3. The oxygenated hemoglobin solution of claim 1, wherein the polymerized hemoglobin includes bovine-derived hemoglobin.

4. The oxygenated hemoglobin solution of claim 1, wherein the polymerized hemoglobin includes hemoglobin polymerized by a dialdehyde.

5. The oxygenated hemoglobin solution of claim 4, wherein the dialdehyde includes glutaraldehyde.

6. The method of claim 1, wherein the tissue is brain, lung, liver, pancreas, spleen, kidney, heart, sections of small intestine, sections of large intestine, rectum, pancreas, skeletal muscles, stomach, urinary bladder, esophagus, larynx, trachea; bronchi, glands including sublingual, parotid submaximal and thyroid glands.

7. The method of claim 1, wherein the vessel is selected from the group consisting of internal carotid, right vertebral, brachiocephalic, sublavian, axillary, dep brachial, brachial, common hepatic, hepatic proper, gastroduodenal, right gastric, right gastroepiploic, superior mesenteric, middle colic, right colic, ileocolic, radial, ulnar, deep palmar arch, superfacial palmer arch, digital, common iliac, internal iliac, external iliac, dorsalis pedis, arcuate, metatarsals, ophthalmic, maximallary, facial, lingual, external carotid, right common carotid, aortic arch, thoracic aorta, abdominal aorta, diaphragm, inferior phrenic celiac trunk, splenic, left gastric, left gastroepiploic, suprarenal, renal, gonadal, left colic, inferior mesenteric, sigmoidal, superior rectal, medial sacral, femoral, popliteal, anterior tibial, posterior tibial, peroneal, plantar arch and digital.

8. The method of claim 1, wherein the vessel is selected from the group consisting of veins of the heart, lung, liver, kidneys, spleen, pancreas, skeletal muscles, urinary bladder, glands, including sublingual, parotid submaximal and thyroid glands.

9. The method of claim 1, wherein the oxygenated hemoglobin solution has a viscosity of between about 1 centipoise and about 2 centipoise at about 37° C.

10. The method of claim 9, wherein the oxygenated hemoglobin solution has a viscosity of about centipoise at about 37° C.

11. The method of claim 9, wherein the P₅₀ of the polymerized hemoglobin is about 40 mm Hg.

12. The method of claim 1, wherein the oxygenated hemoglobin solution includes from about 10 grams to about 250 grams of polymerized hemoglobin per liter of solution, wherein:
a) about 80% by weight, or greater, of the polymerized hemoglobin is oxyhemoglobin;
about 18% by weight, or less, of the polymerized hemoglobin has a molecular weight of over 500,000 Daltons;
c) about 5% by weight, or less, of the polymerized hemoglobin has a molecular weight equal to or less than 65,000 Daltons;
d) a P₅₀ of the polymerized hemoglobin is in a range of between about 34 and about 46 mm Hg; and
e) an endotoxin content of the hemoglobin solution is less than about 0.05 endotoxin units per milliliter.

13. The method of claim 1, wherein the oxygenated hemoglobin solution is administered to the subject at a temperature in a range of between about 18° C. and about 37° C.

14. The method of claim 13, wherein the oxygenated hemoglobin solution is administered to the subject at a temperature in a range of between about 25° C. and about 37° C.

15. The method of claim 13, wherein the oxygenated hemoglobin solution is administered to the subject at a temperature of about 37° C.

16. The method of claim 1, wherein the oxygenated hemoglobin solution is administered by infusion.

17. The method of claim 16, wherein the infusion is an intra-arterial infusion.

18. The method of claim 16, wherein the infusion is a retrograde infusion.

19. The method of claim 16, wherein the oxygenated hemoglobin solution is infused at a rate in a range of between about 10 ml/minute and about 200 ml/minute.

20. The method of claim 19, wherein the oxygenated hemoglobin solution is infused at a rate in a range of between about 20 ml/minute and about 100 ml/minute.

21. The method of claim 20, wherein the infusion rate is in a range of between about 40 ml/minute and about 60 ml/minute.

22. The method of claim 21, wherein the infusion rate is in a range of about 48 ml/minute.

23. The method of claim 1, wherein the oxygenated hemoglobin solution further includes one or more physiological ions.

24. The method of claim 23, wherein the physiological ions include potassium, sodium, calcium and chloride ions.

25. The method of claim 23, wherein the oxygenated hemoglobin solution further comprises glucose, wherein the concentration of glucose is between about 0 and about 50 millimoles per liter.

26. The method of claim 25, wherein the glucose concentration is about 11 millmoles per liter.

27. The method of claim 25, wherein the oxygenated hemoglobin solution further comprises insulin, wherein the concentration of insulin is between about 0 and about 1,000 milliunits per liter.

28. The method of claim 27, wherein the insulin concentration is about 50 milliunits per liter.

29. The method of claim 23, wherein the oxygenated hemoglobin solution further comprises potassium, wherein the concentration of potassium is between about 0 and about 100 millimoles per liter.

30. The method of claim 29, wherein the concentration of potassium is about 4.5 millimoles per liter.

31. A method of treating a patient having ischemia or angina, comprising the step of administering to the subject an oxygenated hemoglobin solution, wherein the oxygenated hemoglobin solution includes polymerized hemoglobin, and wherein about 80% by weight, or greater, of the polymerized hemoglobin is oxyhemoglobin.

32. The method of claim 31, wherein the ischemia or angina is acute.

33. The method of claim 31, wherein the ischemia or angina is caused by an arterial intervention or surgery.

34. The method of claim 33, wherein the ischemia or angina is caused by a cardiac surgery.

35. The method of claim 33, wherein the arterial intervention or surgery is selected from the group consisting of angioplasty; arterial stent deployment; angiography; angioscopy; atherectomy; bypass grafting including coronary and peripheral bypass grafting; endarterectomy; organ transplantation; cardiopulmonary bypass surgery; embolectomy; thrombolytic therapy; aortic surgery; and mesenteric tissue revascularization.

36. The method of claim 31, wherein the ischemia or angina results in a myocardial infarction.

37. The method of claim 31, wherein the oxygenated hemoglobin solution has a viscosity of between about 1 centipoise and about 2 centipoise at about 37° C.

38. The method of claim 36, wherein the oxygenated hemoglobin solution has a viscosity of about 1.3 centipoise at about 37° C.

39. The method of claim 36, wherein the P₅₀ of the polymerized hemoglobin is about 40 mm Hg.

40. The method of claim 31, wherein the oxygenated hemoglobin solution includes from about 10 grams to about 250 grams of polymerized hemoglobin per liter of solution, wherein:
a) about 80% by weight, or greater, of the polymerized hemoglobin is oxyhemoglobin;
b) about 18% by weight, or less, of the polymerized-hemoglobin has a molecular weight of over 500,000 Daltons;
c) about 5% by weight, or less, of the polymerized hemoglobin has a molecular weight equal to or less than 65,000 Daltons;
d) a P₅₀ of the polymerized hemoglobin is in a range of between about 34 and about 46 mm Hg; and
an endotoxin content of the hemoglobin solution is less than about 0.05 endotoxin units per milliliter.

41. The method of claim 40, wherein the oxygenated hemoglobin solution is administered to the subject at a temperature in a range of between about 18° C. and about 37° C.

42. The method of claim 41, wherein the oxygenated hemoglobin solution is administered to the subject at a temperature in a range of between about 25° C. and about 37° C.

43. The method of claim 42, wherein the oxygenated hemoglobin solution is administered to the subject at a temperature of about 37° C.

44. The method of claim 31, wherein the oxygenated hemoglobin solution is administered by infusion.

45. The method of claim 44, wherein the infusion is an intra-arterial infusion.

46. The method of claim 44, wherein the infusion is a retrograde infusion.

47. The method of claim 44, wherein the oxygenated hemoglobin solution is infused at a rate in a range of between about 10 ml/minute and about 200 ml/minute.

48. The method of claim 47, wherein the oxygenated hemoglobin solution is infused at a rate in a range of between about 20 ml/minute and about 100 ml/minute.

49. The method of claim 48, wherein the infusion rate is in a range of between about 40 ml/minute and about 60 ml/minute.

50. The method of claim 49, wherein the infusion rate is in a range of about 48 ml/minute.

51. The method of claim 31, wherein the oxygenated hemoglobin solution further includes one or more physiological ions.

52. The method of claim 51, wherein the physiological ions include potassium, sodium, calcium and chloride ions.

53. The method of claim 52, wherein the oxygenated hemoglobin solution further comprises glucose, wherein the concentration of glucose is between about 0 and about 50 millimoles per liter.

54. The method of claim 53, wherein the glucose concentration is about 11 millmoles per liter.

55. The method of claim 54, wherein the oxygenated hemoglobin solution further comprises insulin, wherein the concentration of insulin is between about 0 and about 1,000 milliunits per liter.

56. The method of claim 55, wherein the insulin concentration is about 50 milliunits per liter.

57. The method of claim 56, wherein the oxygenated hemoglobin solution further comprises potassium, wherein the concentration of potassium is between about 0 and about 100 millimoles per liter.

58. The method of claim 57, wherein the concentration of potassium is about 4.5 millimoles per liter.

59. The method of claim 51, wherein the oxygenated hemoglobin solution further comprises an additional therapeutic agent.

60. The method of claim 59, where in the therapeutic agent is selected from the group consisting of oxfenicine, N⁶-cyclohexyladenosine, mannitol, magnesium, procaine, bicarbonate, tromethamine, mono-sodium L-glutamate monohydrate and monosodium L-aspartate monohydrate, dextrose, glacial acetic acid, citrate, phosphate, dextrose, monobasic sodium phosphate, albumin, sorbitol and aspartate.

61. A plegic solution, appropriate for perfusing one or more different organ types, comprising:
a) a physiological buffer having a pH between about 7.6 and about 7.9;
b) glucose; and
c) polymerized hemoglobin in an amount of between about 10 grams per liter of solution and about 250 grams per liter of solution, wherein
i) about 80% by weight, or greater, of the polymerized hemoglobin is oxyhemoglobin,
ii) about 18% by weight, or less, of the polymerized hemoglobin has a molecular weight of over 500,000 Daltons,
iii) about 5% by weight, or less, of the polymerized hemoglobin has a molecular weight equal to or less than 65,000 Daltons, and
iv) a P₅₀ of the polymerized hemoglobin is in a range of between about 34 and about 46 mm Hg,
wherein an endotoxin content of the plegic solution is less than about 0.05 endotoxin units per milliliter.

62. The plegic solution of claim 61, further comprising an additional therapeutic agent.

63. The plegic solution of claim 62, where in the therapeutic agent is selected from the group consisting of oxfenicine, N⁶-cyclohexyladenosine, mannitol, magnesium, procaine, bicarbonate, tromethamine, mono-sodium L-glutamate monohydrate and monosodium L-aspartate monohydrate, dextrose, glacial acetic acid, citrate, phosphate, dextrose, monobasic sodium-phosphate, albumin, sorbitol and aspartate.

64. The plegic solution of claim 62, wherein the solution has a viscosity of between about 1 centipoise and about 2 centipoise at about 37° C.

65. The plegic solution of claim 64, wherein the solution has a viscosity of between about 1 centipoise and about 1.5 centipoise at about 37° C.

66. The plegic solution of claim 65, wherein the solution has a viscosity of about 1.3 centipoise at about 37° C.

67. The plegic solution of claim 64, wherein the P₅₀ of the polymerized hemoglobin is about 40 mm Hg.

68. The plegic solution of claim 61, wherein the physiological buffer includes at least one component selected from the group consisting of: sodium lactate, N-acetyl-L-cysteine, sodium chloride, potassium chloride and calcium Chloride.2H₂O.

69. An oxygenated hemoglobin solution for use in treating a patient having ischemia or angina, wherein the oxygenated hemoglobin solution includes polymerized hemoglobin, and wherein about 80% by weight, or greater, of the polymerized hemoglobin is oxyhemoglobin.

70. An oxygenated hemoglobin solution packaged and presented for use in treating a patient having ischemia or angina, wherein the oxygenated hemoglobin solution includes polymerized hemoglobin, and wherein about 80% by weight, or greater, of the polymerized hemoglobin is oxyhemoglobin.

71. Use of an oxygenated hemoglobin solution for the manufacture of a medicament for treating a patient having ischemia or angina, wherein the oxygenated hemoglobin solution includes polymerized hemoglobin, and wherein about 80% by weight, or greater, of the polymerized hemoglobin is oxyhemoglobin.
